(19) United States
(12) Patent Application Publication
(10) Pub. No.: US 2026/0248945 A1
(43) Pub. Date: Aug. 27, 2026

(54) MUSCLE TARGETING COMPLEXES AND FORMULATIONS FOR TREATING DYSTROPHINOPATHIES

(71) Applicant: Dyne Therapeutics, Inc., Waltham, MA (US)

(72) Inventors: Timothy Weeden, Waltham, MA (US); Scott Hilderbrand, Cambridge, MA (US); Sean Spring, Waltham, MA (US); Peiyi Shen, Waltham, MA (US); Cody A. Desjardins, Waltham, MA (US); Romesh R. Subramanian, Framingham, MA (US); Mohammed T. Qatanani, Waltham, MA (US); John Najim, Waltham, MA (US); Brendan Quinn, Waltham, MA (US)

(73) Assignee: Dyne Therapeutics, Inc., Waltham, MA (US)

(21) Appl. No.: 18/577,348

(22) PCT Filed: Jul. 8, 2022
(86) PCT No.: PCT/US2022/073540

§ 371 Date: Jan. 8, 2024

Related U.S. Application Data
(60) Provisional application No. 63/220,426, filed on Jul. 9, 2021.

(51) Int. Cl. A61K 47/68; A61P 21/00; C07K 16/28; C07K 19/00

(52) U.S. Cl. CPC A61K 47/6849; A61K 47/6807; A61K 47/6889; A61P 21/00; C07K 16/2881; C07K 19/00

(57) ABSTRACT

Aspects of the disclosure relate to complexes and other aspects relate to formulations (e.g., aqueous, lyophilized forms) comprising such complexes (e.g., wherein each complex is of the exemplary formula shown below) comprising a phosphorodiamidate morpholino oligomer (e.g., useful for targeting DMD) covalently linked to an antibody (e.g., anti-TfR1 antibody). In some embodiments, the complexes are formulated with histidine (e.g., L-histidine) and sucrose at a specified pH (e.g., about 5.0 to 7.0). Also provided are uses of these formulations for treating a subject having a mutated DMD allele associated with Duchenne Muscular Dystrophy. (Formula I)

### RELATED APPLICATIONS

This application claims the benefit under 35 U.S.C § 119(e) of the filing date of U.S. Provisional Application No. 63/220,426, entitled “MUSCLE TARGETING COMPLEXES AND FORMULATIONS FOR TREATING DYSTROPHINOPATHIES”, filed Jul. 9, 2021, the contents of which are incorporated herein by reference in their entirety.

### FIELD OF THE INVENTION

The present application relates to targeting complexes for delivering oligonucleotide molecular payloads to cells, formulations comprising such complexes, and uses thereof, particularly uses relating to treatment of disease.

### REFERENCE TO AN ELECTRONIC SEQUENCE LISTING

The contents of the electronic sequence listing (D082470061W000-SEQ-CBD.xml; Size: 55,409 bytes; and Date of Creation: Jul. 6, 2022) is herein incorporated by reference in its entirety.

### BACKGROUND OF INVENTION

Dystrophinopathies are a group of distinct neuromuscular diseases that result from mutations in DMD gene. Dystrophinopathies include Duchenne muscular dystrophy, Becker muscular dystrophy, and X-linked dilated cardiomyopathy. The DMD, which encodes dystrophin, is a large gene, containing 79 exons and approximately 2.6 million total base pairs. Numerous mutations in DMD, including exonic frameshift, deletion, substitution, and duplicative mutations, are able to diminish the expression of functional dystrophin, leading to dystrophinopathies.

### SUMMARY OF INVENTION

According to some aspects, the present disclosure provides complexes and formulations comprising such complexes. In some embodiments, complexes provided herein are formulated with histidine (e.g., L-histidine) and sucrose. In some embodiments, complexes provided herein are formulated as aqueous or lyophilized (e.g., lyophilized powder) forms. In some embodiments, complexes provided herein are formulated as frozen forms. In some embodiments, complexes provided herein comprise a phosphorodiamidate morpholino oligomer (PMO) covalently linked to an antibody. In some embodiments, complexes provided herein comprise a muscle-targeting complex comprising a PMO covalently linked to an anti-transferrin receptor 1 (TfR1) antibody. In some embodiments, the anti-TfR1 antibody has undergone pyroglutamate formation resulting from a post-translational modification. In some embodiments, a complex comprises a muscle-targeting complex comprising a PMO covalently linked to the anti-transferrin receptor 1 (TfR1) antibody, e.g., having a sequence as set forth in Table 2. In some embodiments, the PMO targets a DMD allele (e.g., a mutated DMD allele). Also provided are methods of using the complexes and formulations described herein for treating a subject having a mutated DMD allele associated with Duchenne Muscular Dystrophy (e.g., wherein the mutated DMD allele comprises a mutation amenable to exon skipping) and/or methods of promoting the expression or activity of a dystrophin protein (e.g., a truncated dystrophin protein) in a cell (e.g., a muscle cell).

Some aspects of the present disclosure provide formulations comprising complexes that comprise a phosphorodiamidate morpholino oligomer (PMO) covalently linked to an anti-transferrin receptor 1 (TfR1) antibody, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, and wherein the complexes are formulated with histidine and sucrose.

Some aspects of the present disclosure provide formulations comprising complexes comprising a structure of formula: [R¹]ₙ₁—R², wherein each R¹ independently comprises a group of the formula (Ta):

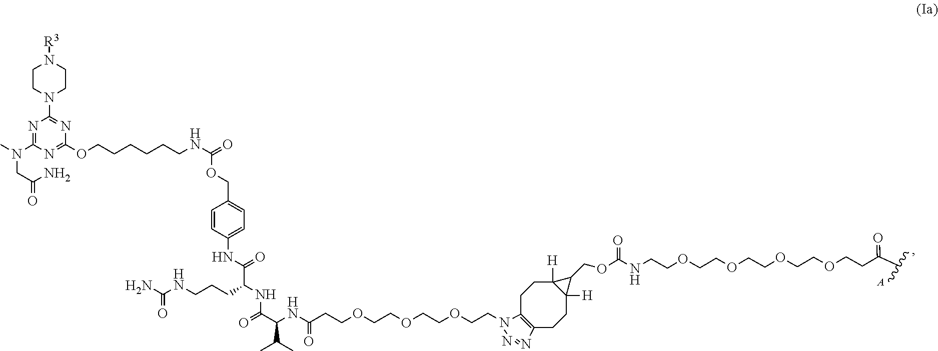

wherein:
R² comprises an antibody, and
R³ comprises a phosphorodiamidate morpholino oligomer (PMO);
wherein R¹ is covalently linked to R² at attachment point A; and
wherein n1 is an integer of one or greater representing the number of instances of R¹,
wherein each instance of R¹ is covalently linked to a different amino acid residue of the antibody;
wherein the complexes are formulated with histidine and sucrose.

In some embodiments, each different amino acid residue is a lysine.

In some embodiments, the antibody is an anti-TfR1 antibody.

In some embodiments, the average value of n1 of complexes in the formulation is in the range of 1 to 5.

In some embodiments, the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16.

In some embodiments, the formulation is in a lyophilized form, an aqueous solution, or a frozen solid form.

In some embodiments, the formulation is in an aqueous solution and the histidine is present in the aqueous solution at a concentration in the range of 10 mM to 50 mM.

In some embodiments, the formulation is in an aqueous solution and the sucrose is present in the aqueous solution at a concentration in the range of 5% to 15% weight per volume (w/v %).

In some embodiments, the formulation is in an aqueous solution and the aqueous solution has a pH in the range of 5.0 to 7.0.

In some embodiments, the formulation is in an aqueous solution and the histidine is present in the aqueous solution at a concentration of 25 mM and/or the sucrose is present in the aqueous solution at a concentration of 10 w/v % and/or the aqueous solution is at a pH of 6.0.

In some embodiments, the antibody is a Fab fragment, a full-length IgG, a Fab′ fragment, a F(ab′)₂ fragment, an scFv, or an Fv.

In some embodiments, the antibody is a Fab fragment.

In some embodiments, the antibody comprises a heavy chain variable region (VH) comprising an amino acid sequence at least 85% identical to SEQ ID NO: 17; and/or wherein the antibody comprises a light chain variable region (VL) comprising an amino acid sequence at least 85% identical to SEQ ID NO: 18.

In some embodiments, the antibody comprises a VH comprising the amino acid sequence of SEQ ID NO: 17 and a VL comprising the amino acid sequence of SEQ ID NO: 18.

In some embodiments, the antibody comprises a heavy chain comprising an amino acid sequence at least 85% identical to SEQ ID NO: 19; and/or wherein the antibody comprises a light chain comprising an amino acid sequence at least 85% identical to SEQ ID NO: 20.

In some embodiments, the antibody comprises a heavy chain comprising the amino acid sequence of SEQ ID NO: 19 and a light chain comprising the amino acid sequence of SEQ ID NO: 20.

In some embodiments, the PMO comprises a nucleobase sequence that is 15-35 nucleotides in length.

In some embodiments, the PMO comprises a nucleotide sequence having a region of complementarity of at least 8 consecutive nucleotides in length to SEQ ID NO: 23, to SEQ ID NO: 24, or to SEQ ID NO: 22.

In some embodiments, the PMO comprises at least 8 consecutive nucleotides of a nucleotide sequence as set forth in SEQ ID NO: 21.

In some embodiments, the PMO comprises the nucleotide sequence of SEQ ID NO: 21.

In some embodiments, each R¹ comprises a group of the formula (Ib):

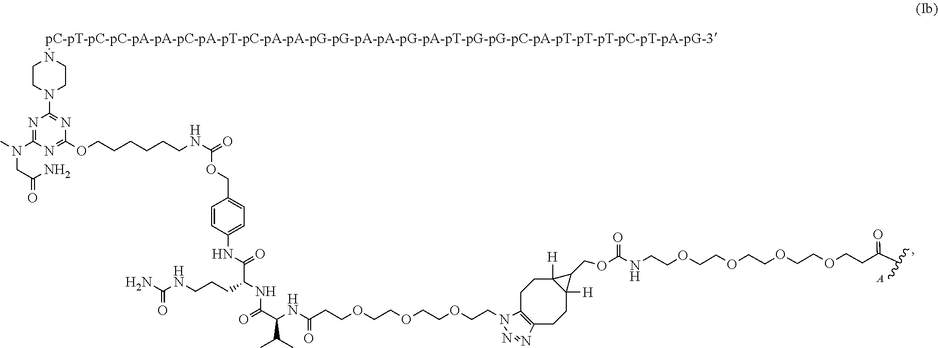

in which -pN indicates a base position of a phosphorodiamidate morpholino oligomer (PMO), wherein -p reflects a phosphorodiamidate linkage, wherein N corresponds to a nucleobase of adenine (A), cytosine (C), guanine (G), or thymine (T), such that the PMO has a nucleobase sequence of CTCCAACATCAAGGAAGATGGCATTTCTAG (SEQ ID NO: 21), and wherein R¹ is covalently linked to R² at attachment point A.

In some embodiments, each R¹ comprises a group of the formula (Ic):

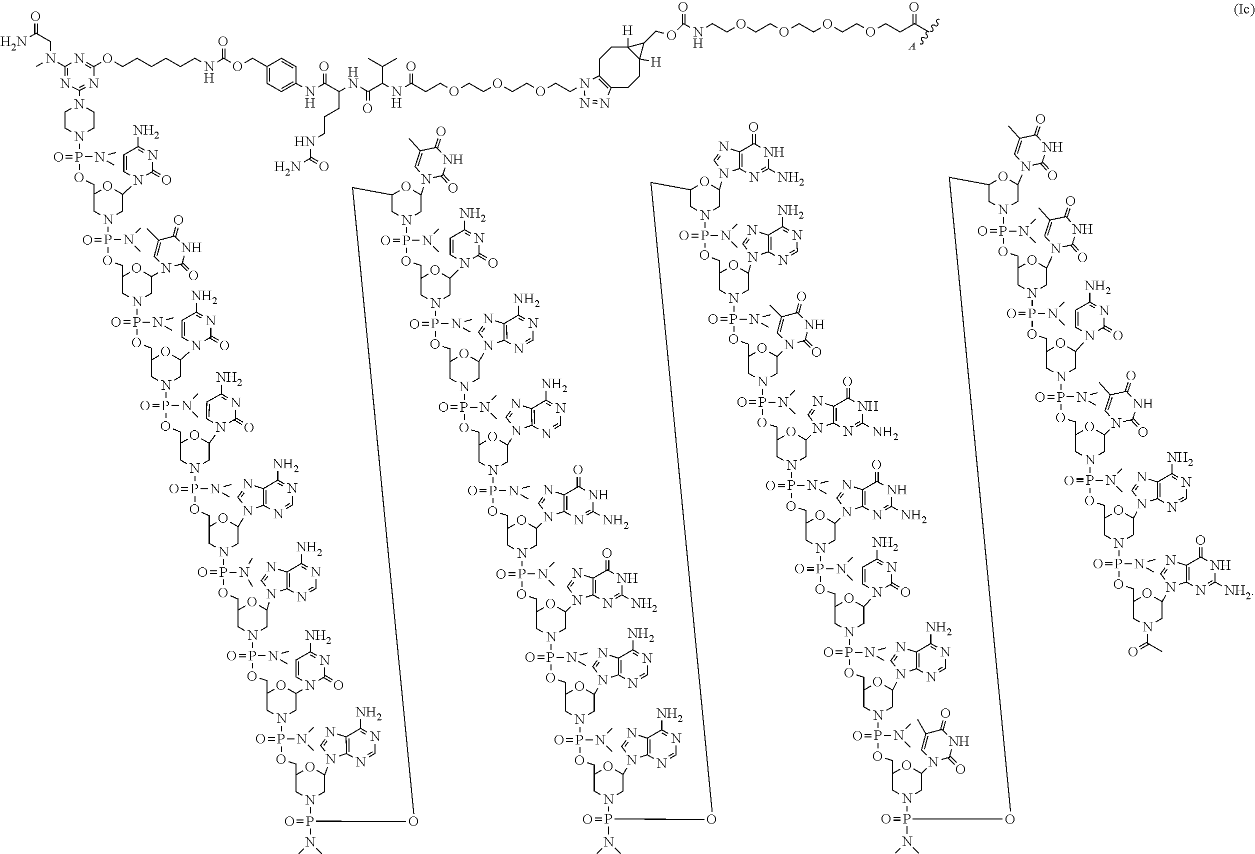

In some embodiments, the complexes are present in the formulation at a concentration in the range of 10 mg/mL to 50 mg/mL.

Further provided herein are methods of promoting expression or activity of a dystrophin protein in a subject, the method comprising administering to the subject the formulation described herein.

In some embodiments, the dystrophin protein is a truncated dystrophin protein.

Further provided herein are methods of treating a subject having a mutated DMD allele associated with Duchenne Muscular Dystrophy, the method comprising administering to the subject the formulation described herein.

In some embodiments, the mutated DMD allele comprises a mutation amenable to exon 51 skipping.

In some embodiments, the mutated DMD allele comprises a frameshift mutation in exon 51.

Other aspects of the present disclosure provide complexes comprising a structure of formula (I): [R¹]ₙ₁—R², wherein
each R¹ comprises a group of the formula (Ia):

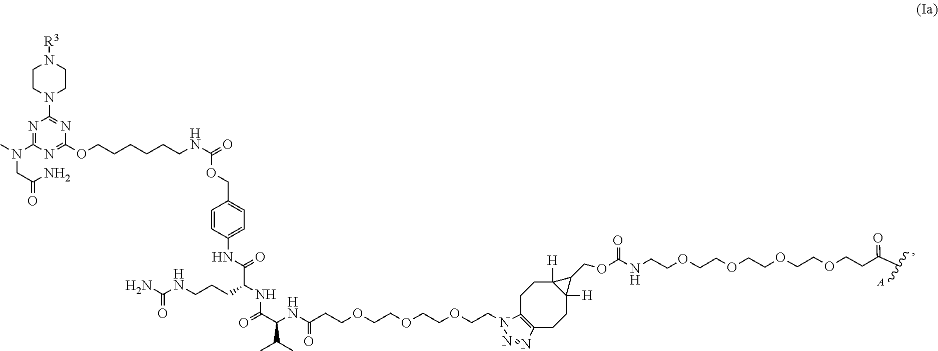

wherein R³ comprises a phosphorodiamidate morpholino oligomer (PMO) comprising the base sequence of CTCCAACATCAAGGAAGATGGCATTTCTAG (SEQ ID NO: 21); wherein R² comprises a Fab, and wherein the Fab comprises a CDR-H1, a CDR-H2, a CDR-H3, a CDR-L1, a CDR-L2, and a CDR-L3 selected from Table 2,
wherein R¹ is covalently linked to R² at attachment point A; and wherein n1 is independently an integer of one or greater representing the number of instances of R¹ in each complex, wherein each instance of R¹ is covalently linked to a different amino acid residue of the Fab.

In some embodiments, the Fab comprises a VH comprising the amino acid sequence of SEQ ID NO: 17 and a VL comprising the amino acid sequence of SEQ ID NO: 18,

In some embodiments, the Fab comprises a heavy chain comprising the amino acid sequence of SEQ ID NO: 19 and a light chain comprising the amino acid sequence of SEQ ID NO: 20.

In some embodiments, each different amino acid residue is a lysine.

Other aspects of the present disclosure provide complexes comprising a structure of formula (I): [R¹]ₙ₁—R², wherein
each R¹ comprises a group of the formula (Ib):

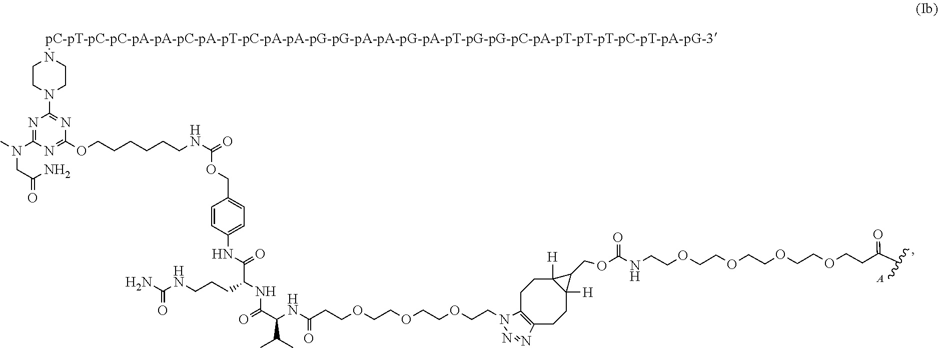

wherein R² comprises a Fab, and wherein the Fab comprises a CDR-H1, a CDR-H2, a CDR-H3, a CDR-L1, a CDR-L2, and a CDR-L3 selected from Table 2,
wherein -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the oligonucleotide PMO has a nucleobase sequence of

(SEQ ID NO: 21)
CTCCAACATCAAGGAAGATGGCATTTCTAG;

wherein R¹ is covalently linked to R² at attachment point A; and wherein n1 is an integer of one or greater representing the number of instances of R¹, wherein each instance of R¹ is covalently linked to a different amino acid residue of the Fab.

In some embodiments, the Fab comprises a VH comprising the amino acid sequence of SEQ ID NO: 17 and a VL comprising the amino acid sequence of SEQ ID NO: 18,

In some embodiments, the Fab comprises a heavy chain comprising the amino acid sequence of SEQ ID NO: 19 and a light chain comprising the amino acid sequence of SEQ ID NO: 20,

In some embodiments, each different amino acid residue is a lysine.

Other aspects of the present disclosure provide complexes comprising a structure of formula (I): [R¹]ₙ₁—R², wherein
each R¹ comprises a group of the formula (Ic):

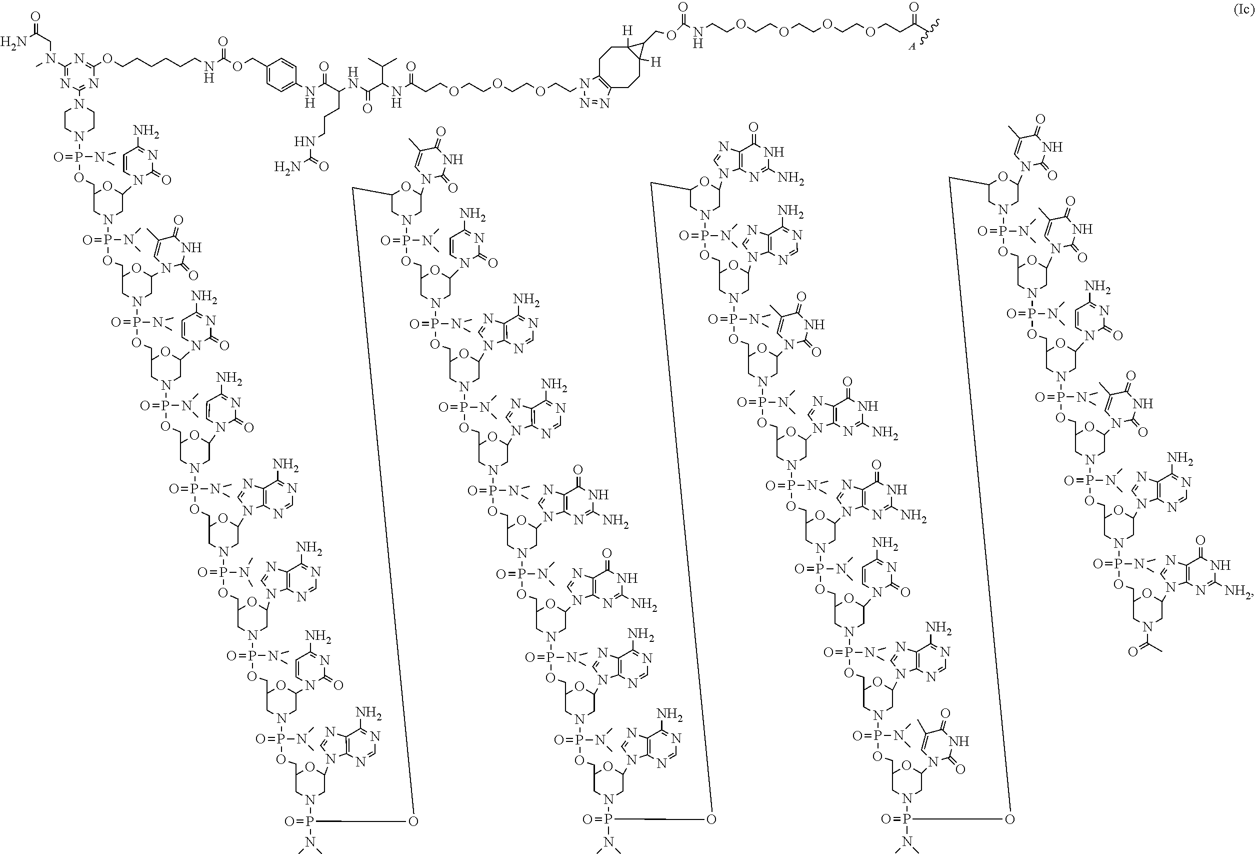

wherein R² comprises a Fab comprising a CDR-H1, a CDR-H2, a CDR-H3, a CDR-L1, a CDR-L2, and a CDR-L3 selected from Table 2;
wherein R¹ is covalently linked to R² at attachment point A; wherein n1 is an integer of one or greater representing the number of instances of R¹, wherein each instance of R¹ is covalently linked to a different amino acid residue of the Fab.

In some embodiments, R² comprises a Fab comprising a VH comprising the amino acid sequence of SEQ ID NO: 17 and a VL comprising the amino acid sequence of SEQ ID NO: 18.

In some embodiments, R² comprises a Fab comprising a heavy chain comprising the amino acid sequence of SEQ ID NO: 19 and a light chain comprising the amino acid sequence of SEQ ID NO: 20.

In some embodiments, each different amino acid residue is a lysine.

Other aspects of the present disclosure provide complexes comprising a structure of formula (Id):

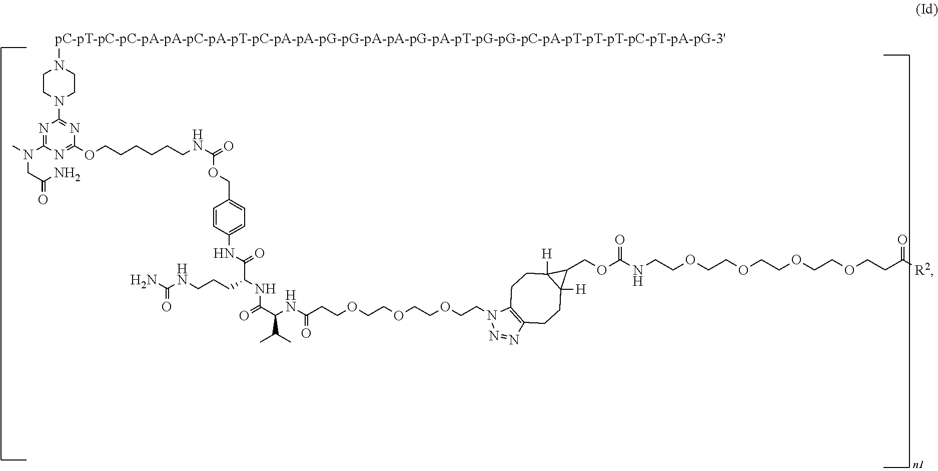

wherein R² comprises a Fab comprising a CDR-H1, a CDR-H2, a CDR-H3, a CDR-L1, a CDR-L2, and a CDR-L3 selected from Table 2;
wherein -pN indicates a base position of a phosphorodiamidate morpholino oligomer (PMO); wherein -p reflects a phosphorodiamidate linkage, and wherein N corresponds to a nucleobase of adenine (A), cytosine (C), guanine (G), or thymine (T), such that the PMO comprises a base sequence of CTCCAACATCAAGGAAGATGGCATTTCTAG (SEQ ID NO: 21);
wherein R¹ is covalently linked to R² at attachment point A; wherein n1 is an integer of one or greater representing the number of instances of R¹, wherein each instance of R¹ is covalently linked to a different amino acid residue of the Fab.

In some embodiments, R² comprises a Fab comprising a VH comprising the amino acid sequence of SEQ ID NO: 17 and a VL comprising the amino acid sequence of SEQ ID NO: 18.

In some embodiments, R² comprises a Fab comprising a heavy chain comprising the amino acid sequence of SEQ ID NO: 19 and a light chain comprising the amino acid sequence of SEQ ID NO: 20.

In some embodiments, each different amino acid residue is a lysine.

### BRIEF DESCRIPTION OF THE DRAWINGS

FIG. 1 shows data illustrating that a composition comprising conjugates comprising an anti-TfR1 Fab having the VH/VL sequences shown in Table 2, in which the Fab is covalently linked (through lysine conjugation) via a linker comprising a valine-citrulline sequence to a DMD exon-skipping oligonucleotide resulted in enhanced exon skipping compared to the naked DMD exon skipping oligo in Duchenne Muscular Dystrophy patient myotubes at a matched equimolar dose of oligonucleotide.

FIG. 2 shows the stability of a composition comprising conjugates comprising an anti-TfR1 Fab having the VH/VL sequences shown in Table 2 covalently linked (through lysine conjugation) via a linker comprising a valine-citrulline sequence to a DMD exon 51-skipping antisense oligonucleotide (ASO) (referred to as “conjugate 1”) in Formulation 1 (25 mg/mL conjugate in 25 mM histidine, 10% sucrose, pH 6) with respect to the concentration of the conjugate over time.

FIG. 3 shows BCA assay results of Formulation 1 comprising the anti-TfR1 Fab having the VH/VL sequences shown in Table 2 covalently linked (through lysine conjugation) via a linker comprising a valine-citrulline sequence to a DMD exon 51-skipping ASO (“conjugate 1”). Once the anti-TfR1 Fab-ASO conjugate 1 was at a 25 mg/mL concentration, samples of the conjugate were added to the desired containers (glass, EVA, PC, HDPE) and frozen at −80° C. overnight, then thawed at 2-8° C. for 4 hours. The standard curves were generated with an R² value of 0.9998. Concentrations of the samples were calculated based on this curve.

FIG. 4 shows the SEC-HPLC results of Formulation 1 comprising the anti-TfR1 Fab having the VH/VL sequences shown in Table 2 covalently linked (through lysine conjugation) via a linker comprising a valine-citrulline sequence to a DMD exon 51-skipping ASO (“anti-TfR1 Fab-ASO conjugate”). There were no material changes in the composition of the peaks across all of the sample formulations. HMWS 1 refers to high molecular weight standard 1 and LMWS 1 refers to low molecular weight standard 1.

### DETAILED DESCRIPTION OF INVENTION

According to some aspects, the present disclosure provides complexes and formulations comprising such complexes. In some embodiments, the complexes are formulated with histidine (e.g., L-histidine) and sucrose. In some embodiments, the complexes are formulated as aqueous or lyophilized (e.g., lyophilized powder) forms. In some embodiments, a complex comprises a phosphorodiamidate morpholino oligomer (PMO) covalently linked to an antibody. In some embodiments, a complex comprises a muscle-targeting complex comprising a PMO covalently linked to an anti-transferrin receptor 1 (TfR1) antibody. In some embodiments, a complex comprises a muscle-targeting complex comprising a PMO covalently linked to the anti-transferrin receptor 1 (TfR1) antibody shown in Table 2. Also provided are methods of using the complexes and formulations described herein for treating a subject having a mutated DMD allele associated with Duchenne Muscular Dystrophy (e.g., wherein the mutated DMD allele comprises a mutation amenable to exon skipping) and/or methods of promoting the expression or activity of a dystrophin protein (e.g., a truncated dystrophin protein) in a cell.

Further aspects of the disclosure, including a description of defined terms, are provided below.

### Definitions

Administering: As used herein, the terms “administering” or “administration” means to provide a complex to a subject in a manner that is physiologically and/or (e.g., and) pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a Fab′ fragment, a F(ab′)₂ fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information System® http://www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. Examples of CDR definition systems are provided in Table 1.

TABLE 1
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

DMD: As used herein, the term “DMD” refers to a gene that encodes dystrophin protein, a key component of the dystrophin-glycoprotein complex, which bridges the inner cytoskeleton and the extracellular matrix in muscle cells, particularly muscle fibers. Deletions, duplications, and point mutations in DMD may cause dystrophinopathies, such as Duchenne muscular dystrophy, Becker muscular dystrophy, or cardiomyopathy. Alternative promoter usage and alternative splicing result in numerous distinct transcript variants and protein isoforms for this gene. In some embodiments, a DMD gene may be a human (Gene ID: 1756), non-human primate (e.g., Gene ID: 465559), or rodent gene (e.g., Gene ID: 13405; Gene ID: 24907). In addition, multiple human transcript variants (e.g., as annotated under GenBank RefSeq Accession Numbers: NM_000109.3, NM_004006.2 (SEQ ID NO: 24), NM_004009.3, NM_004010.3 and NM_004011.3) have been characterized that encode different protein isoforms.

DMD allele: As used herein, the term “DMD allele” refers to any one of alternative forms (e.g., wild-type or mutant forms) of a DMD gene. In some embodiments, a DMD allele may encode for dystrophin that retains its normal and typical functions. In some embodiments, a DMD allele may comprise one or more mutations that results in muscular dystrophy. Common mutations that lead to Duchenne muscular dystrophy involve frameshift, deletion, substitution, and duplicative mutations of one or more of 79 exons present in a DMD allele, e.g., exon 8, exon 23, exon 41, exon 44, exon 50, exon 51, exon 52, exon 53, or exon 55. Further examples of DMD mutations are disclosed, for example, in Flanigan K M, et al., Mutational spectrum of DMD mutations in dystrophinopathy patients: application of modern diagnostic techniques to a large cohort. Hum Mutat. 2009 December; 30 (12):1657-66, the contents of which are incorporated herein by reference in its entirety.

Dystrophinopathy: As used herein, the term “dystrophinopathy” refers to a muscle disease that results from one or more mutated DMD alleles. Dystrophinopathies include a spectrum of conditions (ranging from mild to severe) that includes Duchenne muscular dystrophy, Becker muscular dystrophy, and DMD-associated dilated cardiomyopathy (DCM). In some embodiments, at one end of the spectrum, dystrophinopathy is phenotypically associated with an asymptomatic increase in serum concentration of creatine phosphokinase (CK) and/or (e.g., and) muscle cramps with myoglobinuria. In some embodiments, at the other end of the spectrum, dystrophinopathy is phenotypically associated with progressive muscle diseases that are generally classified as Duchenne or Becker muscular dystrophy when skeletal muscle is primarily affected and as DMD-associated dilated cardiomyopathy (DCM) when the heart is primarily affected. Symptoms of Duchenne muscular dystrophy include muscle loss or degeneration, diminished muscle function, pseudohypertrophy of the tongue and calf muscles, higher risk of neurological abnormalities, and a shortened lifespan. Duchenne muscular dystrophy is associated with Online Mendelian Inheritance in Man (OMIM) Entry #310200. Becker muscular dystrophy is associated with OMIM Entry #300376. Dilated cardiomyopathy is associated with OMIM Entry X #302045.

Exonic splicing enhancer (ESE): As used herein, the term “exonic splicing enhancer” or “ESE” refers to a nucleic acid sequence motif within an exon of a gene, pre-mRNA, or mRNA that directs or enhances splicing of pre-mRNA into mRNA, e.g., as described in Blencowe et al., Trends Biochem Sci 25, 106-10. (2000), incorporated herein by reference. ESEs are splicing features. ESEs may direct or enhance splicing, for example, to remove one or more introns and/or one or more exons from a gene transcript. ESE motifs are typically 6-8 nucleobases in length. SR proteins (e.g., proteins encoded by the gene SRSF1, SRSF2, SRSF3, SRSF4, SRSF5, SRSF6, SRSF7, SRSF8, SRSF9, SRSF10, SRSF11, SRSF12, TRA2A or TRA2B) bind to ESEs through their RNA recognition motif region to facilitate splicing. ESE motifs can be identified through a number of methods, including those described in Cartegni et al., Nucleic Acids Research, 2003, Vol. 31, No. 13, 3568-3571, incorporated herein by reference.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti-transferrin receptor antibodies and antigen binding portions are provided. Such antibodies may be generated by obtaining murine anti-transferrin receptor monoclonal antibodies using traditional hybridoma technology followed by humanization using in vitro genetic engineering, such as those disclosed in Kasaian et al PCT publication No. WO 2005/123126 A2.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Morpholinos: As used herein, the term “morpholino”, also referred to as a “phosphorodiamidate morpholino oligomer”, refers to a molecular structure that contains nucleobases attached to a backbone of methylenemorpholine rings linked through a phosphorodiamidate group. In some embodiments, the oligonucleotide may be a morpholino-based compounds. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidate morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleosides (e.g., 2′-O-methyl sugar modifications, purine or pyrimidine modifications). In some embodiments, an oligonucleotide may comprise one or more modified internucleoside linkage. In some embodiments, an oligonucleotide may comprise one or more phosphorothioate linkages, which may be in the Rp or Sp stereochemical conformation.

Region of complementarity: As used herein, the term “region of complementarity” refers to a nucleotide sequence, e.g., of an oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a disease resulting from a mutated DMD gene sequence, e.g., a mutation in an exon of a DMD gene sequence. In some embodiments, a subject has a dystrophinopathy, e.g., Duchenne muscular dystrophy.

Transferrin receptor: As used herein, the term, “transferrin receptor” (also known as TFRC, CD71, p90, TFR, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

Ranges: All ranges provided in the present disclosure are inclusive of the end points.

### Complexes

Provided herein are complexes that comprise a targeting agent, e.g., an antibody, covalently linked to an oligonucleotide. In some embodiments, a complex comprises a muscle-targeting antibody covalently linked to one or more oligonucleotides. In some embodiments, the oligonucleotide is a PMO. In some embodiments, the oligonucleotide is an oligonucleotide that targets a mutated DMD allele to promote exon skipping.

Complexes described herein generally comprise a linker that covalently links an antibody (e.g., any one of the anti-TfR1 antibodies) described herein to an oligonucleotide (e.g., a PMO). A linker comprises at least one covalent bond.

In some embodiments, complexes provided herein (e.g., in compositions or formulations described herein) comprise a structure of formula (I): [R¹]ₙ₁—R², in which each R¹ independently comprises a compound comprising an oligonucleotide (e.g., a PMO) and R² comprises an antibody (e.g., anti-TfR1 antibody), and in which n1 is an integer (e.g., of one or greater) representing the number of instances of R¹ in the complex. In some embodiments, in each complex n1 is independently an integer (e.g., of zero or greater) representing the number of instances of R¹ in each complex. In some embodiments, each R¹ independently comprises a group comprising an oligonucleotide. In some embodiments, each R¹ independently comprises a group that comprises additional elements in addition to an oligonucleotide. In some embodiments, R² comprises an antibody (e.g., anti-TfR1 antibody) comprising a heavy chain comprising a heavy chain variable region (VH) and a heavy chain constant region, and a light chain comprising a light chain variable region (VL) and a light chain constant region. In some embodiments, each R¹ of a complex is independently covalently linked to a different amino acid residue (e.g., lysine or cysteine) of R². In some embodiments, R² comprises an anti-TfR1 Fab.

In some embodiments, in each complex, n1 is independently an integer of zero or greater. In some embodiments, in each complex, n1 is independently an integer of one or greater. In some embodiments, n1 is an integer of one or greater. In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)2 fragment, an scFv, or an Fv. In some embodiments, the antibody is a Fab fragment.

In some embodiments, the value of n1 of each or any complex (e.g., any complex in any of the compositions or formulations disclosed herein) is an integer from one up to the number of amino acid residues in the antibody to which conjugation is desired or targeted (e.g., the number of lysine residues). In some embodiments, the value of n1 is selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, the value of n1 is selected from 1, 2, 3, 4, 5, 6, 7, 8, 9, 10, 11, 12, 13, 14, 15, 16, 17, 18, 19, 20, 21, 22, 23, 24, 25 and 26. In some embodiments, the value of n1 is in the range of 1-27, 1-26, 1-10, 1-5, or 1-3. In some embodiments, in each complex, the value of n1 is independently selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, in each complex, the value of n1 is independently selected from 1, 2, 3, 4, 5, 6, 7, 8, 9, 10, 11, 12, 13, 14, 15, 16, 17, 18, 19, 20, 21, 22, 23, 24, 25 and 26. In some embodiments, in each complex, the value of n1 is independently in the range of 1-27, 1-26, 1-10, 1-5, or 1-3. In some embodiments, the average value of n1 of complexes of the composition is in the range of 1 to 5 (e.g., 1-5, 1-4, 1-3, 3-5, or 1-2). In some embodiments, compositions described herein comprise complexes that comprise a structure of formula (I): [R¹]ₙ₁—R², wherein n1 is 0. In some embodiments, the average value of n1 of complexes of the composition is in the range of 1 to 5 (e.g., 1-5, 1-4, 1-3, 1-2, 2-4, 3-5, 1-4.6, 1-4.5, 1-4.4, 1-4.3, 1-4.2, 1-3.5, 1-2.5, 1.1-5, 1.1-4.5, 1.1-4, 1.1-3.5, 1.1-3, 1.1-2.5, 1.1-2.2, 1.2-5, 1.2-4.5, 1.2-4, 1.2-3.5, 1.2-3, 1.2-2.5, 1.2-2.2, 1.3-5, 1.3-4.5, 1.3-4, 1.3-3.5, 1.3-3, 1.3-2.5, 1.3-2.2, 1.4-5, 1.4-4.5, 1.4-4, 1.4-3.5, 1.4-3, 1.4-2.5, 1.4-2.2, 1.5-5, 1.5-4.5, 1.5-4, 1.5-3.5, 1.5-3, 1.5-2.5, 1.5-2.2, 1.6-3, 1.6-2.5, 1.6-2.2, 1.7-3, 1.7-2.5, 1.7-2.2, 1.8-3, 1.8-2.5, or 1.8-2.2). In some embodiments, in each complex type n1 is independently an integer of one or greater representing the number of instances of R¹ in each complex of the complex type, and in which the different complex types of the composition are characterized by having different n1 values (e.g., n1 values in the range of 1-27, 1-26, 1-25, 1-20, 1-15, 1-10, 1-5, or 1-3).

In some embodiments, compositions are provided (e.g., formulations comprising histidine and/or sucrose, as described herein) that comprise a plurality of different complexes. In some embodiments, the plurality of different complexes comprise a common targeting agent (e.g. an antibody) and a common oligonucleotide (e.g., PMO). In such embodiments, different complex types are characterized by having different numbers of oligonucleotides covalently linked to an antibody. For example, in some embodiments, compositions are provided that comprise a plurality of complex types in which each complex type comprises a structure of formula (I): [R¹]ₙ₁—R², in which each R¹ independently comprises a compound comprising an oligonucleotide (e.g., a PMO) and R² comprises an antibody (e.g., anti-TfR1 antibody), and in which in each complex type n1 is independently is an integer of one or greater representing the number of instances of R¹ in each complex of the complex type, and in which the different complex types of the composition are characterized by having different n1 values (e.g., n1 values in the range of 1-27, 1-26). In some embodiments, each different complex types of the composition have different n1 values in the range of 1-27, 1-26, 1-25, 1-20, 1-15, 1-10, 1-5, or 1-3. In some embodiments, in complexes of a composition n1 is independently an integer. In some embodiments, the average value of n1 of complexes of the composition is in the range of 1 to 5 (e.g., 1-5, 1-4, 1-3, 1-2, 2-4, 3-5, 1-4.6, 1-4.5, 1-4.4, 1-4.3, 1-4.2, 1-3.5, 1-2.5, 1.1-5, 1.1-4.5, 1.1-4, 1.1-3.5, 1.1-3, 1.1-2.5, 1.1-2.2, 1.2-5, 1.2-4.5, 1.2-4, 1.2-3.5, 1.2-3, 1.2-2.5, 1.2-2.2, 1.3-5, 1.3-4.5, 1.3-4, 1.3-3.5, 1.3-3, 1.3-2.5, 1.3-2.2, 1.4-5, 1.4-4.5, 1.4-4, 1.4-3.5, 1.4-3, 1.4-2.5, 1.4-2.2, 1.5-5, 1.5-4.5, 1.5-4, 1.5-3.5, 1.5-3, 1.5-2.5, 1.5-2.2, 1.6-3, 1.6-2.5, 1.6-2.2, 1.7-3, 1.7-2.5, 1.7-2.2, 1.8-3, 1.8-2.5, or 1.8-2.2). In some embodiments, compositions described herein comprise complexes in which n1 is 0.

In some embodiments, compositions are provided herein (e.g., formulations comprising histidine and/or sucrose as described herein) that comprise unconjugated antibody (e.g., in trace amounts) and antibody conjugated to one or more oligonucleotides. As used herein, an “unconjugated antibody” refers to an antibody that is not conjugated to an oligonucleotide. In some embodiments, unconjugated antibody may be referred to as a compound comprising a structure of formula (I): [R¹]ₙ₁—R², for which n1 is zero. Accordingly, in some embodiments, compositions are provided (e.g., formulations as described herein) that comprise compounds (e.g., complexes) comprising a structure of formula (I): [R¹]ₙ₁—R², for which each R¹ independently comprises a group comprising an oligonucleotide, R² comprises an antibody and n1 is an integer of zero or greater that reflects the number of instances of R¹ in the complex. In some embodiments, n1 is independently an integer of zero or greater that reflects the number of instances of R¹ in each compound (e.g., complex). In some embodiments, the fraction of compounds comprising a structure of formula (I): [R¹]ₙ₁—R², in a composition, for which n1 is zero, compared with all compounds of that structure in the composition for which n1 is one or greater, is less than 10%, less than 5%, less than 1% less than 0.5%, less than 0.1%, less than 0.05%, or less than 0.01%. As such, in some embodiments, the average value of n1 of complexes of the composition is in the range of 1 to 5 (e.g., 1-5, 1-4, 1-3, 1-2, 2-4, 3-5, 1-4.6, 1-4.5, 1-4.4, 1-4.3, 1-4.2, 1-3.5, 1-2.5, 1.1-5, 1.1-4.5, 1.1-4, 1.1-3.5, 1.1-3, 1.1-2.5, 1.1-2.2, 1.2-5, 1.2-4.5, 1.2-4, 1.2-3.5, 1.2-3, 1.2-2.5, 1.2-2.2, 1.3-5, 1.3-4.5, 1.3-4, 1.3-3.5, 1.3-3, 1.3-2.5, 1.3-2.2, 1.4-5, 1.4-4.5, 1.4-4, 1.4-3.5, 1.4-3, 1.4-2.5, 1.4-2.2, 1.5-5, 1.5-4.5, 1.5-4, 1.5-3.5, 1.5-3, 1.5-2.5, 1.5-2.2, 1.6-3, 1.6-2.5, 1.6-2.2, 1.7-3, 1.7-2.5, 1.7-2.2, 1.8-3, 1.8-2.5, or 1.8-2.2).

In some embodiments, each instance of R¹ in a complex is conjugated to a different amino acid residue of the antibody. In some embodiments, each instance of R¹ in a complex is covalently linked to a different amino acid residue of the antibody. In some embodiments, an amino acid to which R¹ is covalently linked comprises an F-amino group (e.g., lysine, arginine). In some embodiments, each different amino acid comprises an F-amino group (e.g., lysine, arginine). However, in some embodiments, an amino acid to which R¹ is covalently linked is a cysteine. In some embodiments, each different amino acid to which R¹ is covalently linked is a cysteine. In some embodiments, R¹ is directly covalently linked to an amino acid residue of the antibody. However, in some embodiments, R¹ is indirectly covalently linked to an amino acid of the antibody, e.g., covalently linked to a glycosylation site on the amino acid.

In some embodiments, R¹ is not covalently linked to an amino acid residue residing in a CDR region of the antibody.

In some embodiments, complexes provided herein (e.g., in compositions or formulations described herein) comprise a structure of formula (I): [R¹]ₙ₁—R², in which each instance of R¹ independently comprises a group of the formula (Ia):

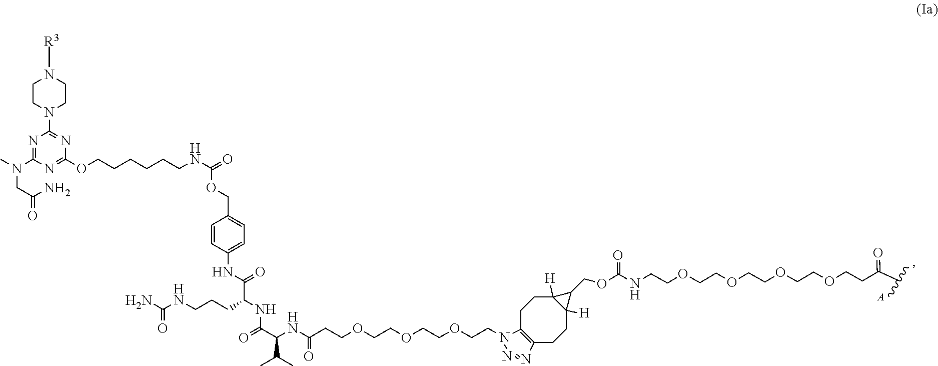

in which R comprises an oligonucleotide, e.g., a phosphorodiamidate morpholino oligomer (PMO); and R¹ is covalently linked (e.g., indirectly or directly linked, e.g., directly linked) to R² at attachment point A. In some embodiments, in each complex n1 is independently an integer (e.g., of one or greater) representing the number of instances of R¹ in each complex. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, R³ comprises an oligonucleotide, e.g., a phosphorodiamidate morpholino oligomer (PMO) comprising the base sequence of CTCCAACATCAAGGAAGATGGCATTTCTAG (SEQ ID NO: 21). In some embodiments, R² comprises a Fab and each R¹ is covalently linked at attachment point A to a different amino acid residue of the Fab, optionally wherein each different amino acid residue is a lysine. In some embodiments, in each complex n1 is independently an integer (e.g., an integer in the range of 1-27, 1-26, 1-10, 1-5, or 1-3).

In some embodiments, complexes provided herein (e.g., in compositions or formulations described herein) comprise a structure of formula (I): [R¹]ₙ₁— R², in which each R¹ comprises a group of the formula (Ib):

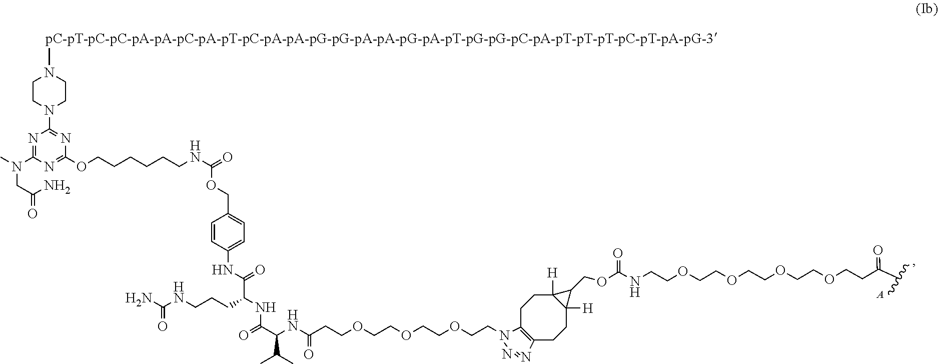

in which -pN indicates a base position of a phosphorodiamidate morpholino oligomer (PMO); R¹ is covalently linked (e.g., indirectly or directly linked, e.g., directly linked) to R² at attachment point A, wherein -p reflects a phosphorodiamidate linkage, and wherein N corresponds to a nucleobase of adenine (A), cytosine (C), guanine (G), or thymine (T), such that the PMO comprises a base sequence of CTCCAACATCAAGGAAGATGGCATTTCTAG (SEQ ID NO: 21). In some embodiments, in each complex n1 is independently an integer (e.g., of one or greater) representing the number of instances of R¹ in each complex, and each R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18.

In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, R² comprises a Fab and each R¹ is covalently linked at attachment point A to a different amino acid residue of the Fab, optionally wherein each different amino acid residue is a lysine.

In some embodiments, complexes provided herein (e.g., in compositions or formulations described herein) comprise a structure of formula (I): [R¹]ₙ₁— R², in which each R¹ comprises a group of the formula (Ic):

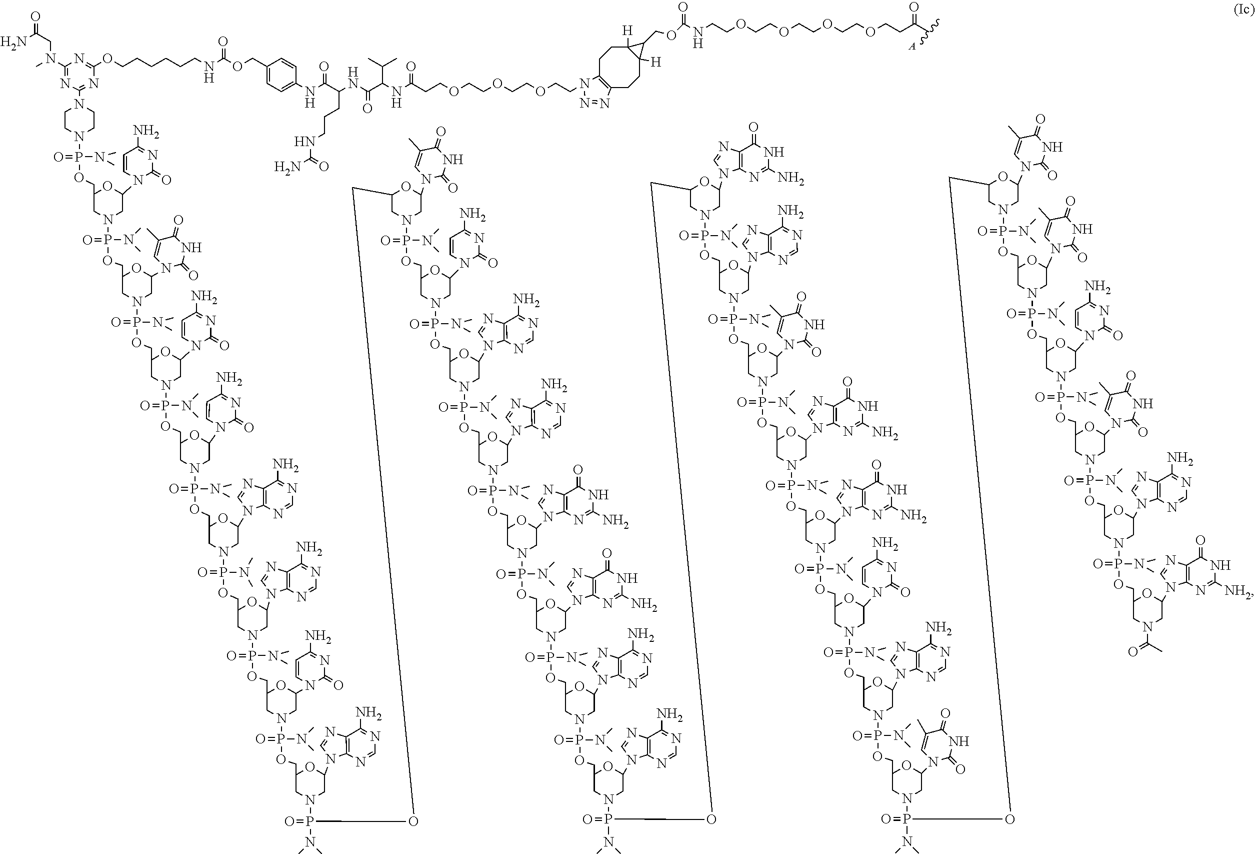

wherein R¹ is covalently linked (e.g., indirectly or directly linked, e.g., directly linked) to R² at attachment point A. In some embodiments, in each complex n1 is independently an integer (e.g., of one or greater) representing the number of instances of R¹ in each complex, wherein each R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, R² comprises a Fab and each R¹ is covalently linked at attachment point A to a different amino acid residue of the Fab, optionally wherein each different amino acid residue is a lysine.

In some embodiments, complexes provided herein (e.g., in compositions or formulations described herein) comprise a structure of the formula (Id):

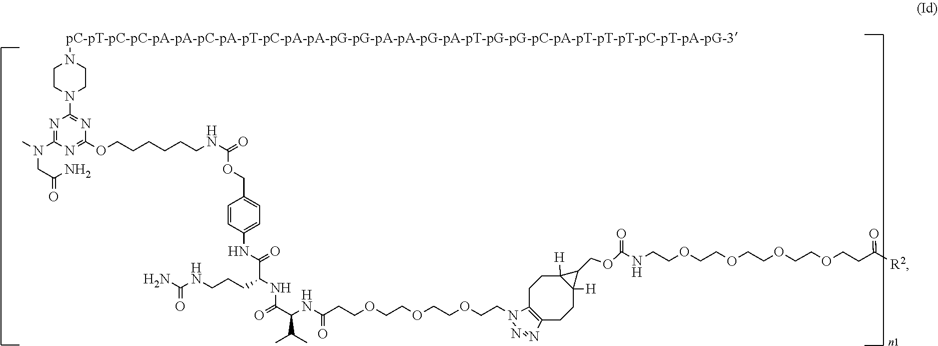

in which -pN indicates a base position of a phosphorodiamidate morpholino oligomer (PMO); wherein -p reflects a phosphorodiamidate linkage, and wherein N corresponds to a nucleobase of adenine (A), cytosine (C), guanine (G), or thymine (T), such that the PMO comprises a base sequence of CTCCAACATCAAGGAAGATGGCATTTCTAG (SEQ ID NO: 21); wherein R² comprises an antibody comprising a sequence as set forth in Table 2; wherein in each complex n1 is independently an integer (e.g., of one or greater) representing the number of instances of the group enclosed by square brackets, wherein each instance of the group enclosed by square brackets is covalently linked to a different amino acid residue of the antibody (e.g., a Fab), optionally wherein each different amino acid residue is a lysine. In some embodiments, R² comprises an antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody (e.g., a Fab) comprising a VH comprising the amino acid sequence of SEQ ID NO: 17 and/or comprising a VL comprising the amino acid sequence of SEQ ID NO: 18. In some embodiments, R² comprises an antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody (e.g., a Fab) comprising a heavy chain comprising the amino acid sequence of SEQ ID NO: 19 and/or comprising a light chain comprising the amino acid sequence of SEQ ID NO: 20. In some embodiments, in each complex n1 is independently an integer (e.g., an integer in the range of 1-27, 1-26, 1-10, 1-5, or 1-3). In some embodiments, R² comprises an antibody (e.g., a Fab) that is covalently linked via different amino acid residue of the antibody (e.g., Fab), optionally wherein each different amino acid residue is a lysine.

In some embodiments, complexes described herein comprise a structure of:

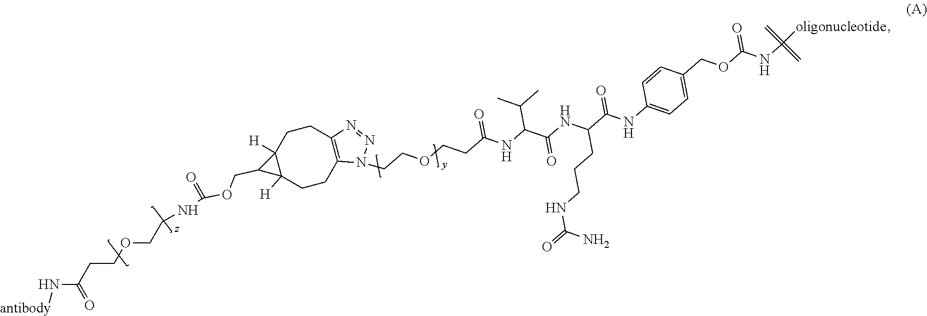

wherein y is 0-15 (e.g., 3) and z is 0-15 (e.g., 4). In some embodiments, the antibody is an anti-TfR1 antibody (e.g., the anti-TfR1 antibody provided in Table 2). In some embodiments, the oligonucleotide is a PMO and comprises the nucleotide sequence of SEQ ID NO: 21. In some embodiments, the amide shown adjacent to the anti-TfR1 antibody in the structure (A) results from a reaction with an amine of the anti-TfR1 antibody, such as a lysine epsilon amine. In some embodiments, a complex described herein comprises an anti-TfR1 Fab covalently linked via a lysine of the Fab to the 5′ end of a PMO. In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)2 fragment, an scFv, or an Fv.

### Antibodies

In some embodiments, complexes provided herein comprise an antibody that binds human transferrin receptor 1 (TfR1). An example human transferrin receptor 1 amino acid sequence, corresponding to NCBI sequence NP_003225.2 (transferrin receptor protein 1 isoform 1, Homo sapiens) is as follows:

(SEQ ID NO: 35)
MMDQARSAFSNLFGGEPLSYTRFSLARQVDGDNSHVEMKLAVDEEENADN
NTKANVTKPKRCSGSICYGTIAVIVFFLIGFMIGYLGYCKGVEPKTECER
LAGTESPVREEPGEDFPAARRLYWDDLKRKLSEKLDSTDFTGTIKLLNEN
SYVPREAGSQKDENLALYVENQFREFKLSKVWRDQHFVKIQVKDSAQNSV
IIVDKNGRLVYLVENPGGYVAYSKAATVTGKLVHANFGTKKDFEDLYTPV
NGSIVIVRAGKITFAEKVANAESLNAIGVLIYMDQTKFPIVNAELSFFGH
AHLGTGDPYTPGFPSFNHTQFPPSRSSGLPNIPVQTISRAAAEKLFGNME
GDCPSDWKTDSTCRMVTSESKNVKLTVSNVLKEIKILNIFGVIKGFVEPD
HYVVVGAQRDAWGPGAAKSGVGTALLLKLAQMFSDMVLKDGFQPSRSIIF
ASWSAGDFGSVGATEWLEGYLSSLHLKAFTYINLDKAVLGTSNFKVSASP
LLYTLIEKTMQNVKHPVTGQFLYQDSNWASKVEKLTLDNAAFPFLAYSGI
PAVSFCFCEDTDYPYLGTTMDTYKELIERIPELNKVARAAAEVAGQFVIK
LTHDVELNLDYERYNSQLLSFVRDLNQYRADIKEMGLSLOWLYSARGDFF
RATSRLTTDFGNAEKTDRFVMKKLNDRVMRVEYHFLSPYVSPKESPFRHV
FWGSGSHTLPALLENLKLRKQNNGAFNETLFRNQLALATWTIQGAANALS
GDVWDIDNEF.

Table 2 provides examples of sequences of an anti-TfR1 antibody useful in the complexes provided herein.

TABLE 2
Examples of anti-TfR1 antibody sequences

| antibody | IMGT | Kabat | Chothia |
|---|---|---|---|
| CDR-H1 | GYSITSGYY | SGYYWN (SEQ ID | GYSITSGY (SEQ ID NO: 12) |
| | (SEQ ID NO: 1) | NO: 7) | |
| CDR-H2 | ITFDGAN | YITFDGANNYNPSL | FDG (SEQ ID NO: 13) |
| | (SEQ ID NO: 2) | KN (SEQ ID NO: 8) | |
| CDR-H3 | TRSSYDYDVL | SSYDYDVLDY | SYDYDVLD (SEQ ID NO: 14) |
| | DY (SEQ ID | (SEQ ID NO: 9) | |
| | NO: 3) | | |
| CDR-L1 | QDISNF (SEQ | RASQDISNFLN | SQDISNF (SEQ ID NO: 15) |
| | ID NO: 4) | (SEQ ID NO: 10) | |
| CDR-L2 | YTS (SEQ ID | YTSRLHS (SEQ ID | YTS (SEQ ID NO: 5) |
| | NO: 5) | NO: 11) | |
| CDR-L3 | QQGHTLPYT | QQGHTLPYT (SEQ | GHTLPY (SEQ ID NO: 16) |
| | (SEQ ID NO: 6) | ID NO: 6) | |

| | |
|---|---|
| VH | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSS (SEQ ID NO: 17) |
| VL | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTFG |
| | QGTKLEIK (SEQ ID NO: 18) |
| Fab HC | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSSASTKGPSVFPLAPSSKSTSGGTAALGC |
| | LVKDYFPEPVTVSWNSGALTSGVHTFPAVLQSSGLYSLSSVVTVPSSSL |
| | GTQTYICNVNHKPSNTKVDKKVEPKSCDKTHT (SEQ ID NO: 19) |
| Fab LC | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTFG |
| | QGTKLEIKRTVAAPSVFIFPPSDEQLKSGTASVVCLLNNFYPREAKVQW |
| | KVDNALQSGNSQESVTEQDSKDSTYSLSSTLTLSKADYEKHKVYACEV |
| | THQGLSSPVTKSFNRGEC (SEQ ID NO: 20) |

In some embodiments, the anti-TfR1 antibody of the present disclosure comprises a heavy chain complementarity determining region 1 (CDR-H1) of SEQ ID NO: 1 (according to the IMGT definition system), a heavy chain complementarity determining region 2 (CDR-H2) of SEQ ID NO: 2 (according to the IMGT definition system), a heavy chain complementarity determining region 3 (CDR-H3) of SEQ ID NO: 3 (according to the IMGT definition system), a light chain complementarity determining region 1 (CDR-L1) of SEQ ID NO: 4 (according to the IMGT definition system), a light chain complementarity determining region 2 (CDR-L2) of SEQ ID NO: 5 (according to the IMGT definition system), and a light chain complementarity determining region 3 (CDR-L3) of SEQ ID NO: 6 (according to the IMGT definition system).

In some embodiments, the anti-TfR1 antibody of the present disclosure comprises a heavy chain complementarity determining region 1 (CDR-H1) of SEQ ID NO: 7 (according to the Kabat definition system), a heavy chain complementarity determining region 2 (CDR-H2) of SEQ ID NO: 8 (according to the Kabat definition system), a heavy chain complementarity determining region 3 (CDR-H3) of SEQ ID NO: 9 (according to the Kabat definition system), a light chain complementarity determining region 1 (CDR-L1) of SEQ ID NO: 10 (according to the Kabat definition system), a light chain complementarity determining region 2 (CDR-L2) of SEQ ID NO: 11 (according to the Kabat definition system), and a light chain complementarity determining region 3 (CDR-L3) of SEQ ID NO: 6 (according to the Kabat definition system).

In some embodiments, the anti-TfR1 antibody of the present disclosure comprises a heavy chain complementarity determining region 1 (CDR-H1) of SEQ ID NO: 12 (according to the Chothia definition system), a heavy chain complementarity determining region 2 (CDR-H2) of SEQ ID NO: 13 (according to the Chothia definition system), a heavy chain complementarity determining region 3 (CDR-H3) of SEQ ID NO: 14 (according to the Chothia definition system), a light chain complementarity determining region 1 (CDR-L1) of SEQ ID NO: 15 (according to the Chothia definition system), a light chain complementarity determining region 2 (CDR-L2) of SEQ ID NO: 5 (according to the Chothia definition system), and a light chain complementarity determining region 3 (CDR-L3) of SEQ ID NO: 16 (according to the Chothia definition system).

In some embodiments, the anti-TfR1 antibody of the present disclosure comprises a heavy chain variable region (VH) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH comprising the amino acid sequence of SEQ ID NO: 17. Alternatively or in addition (e.g., in addition), the anti-TfR1 antibody of the present disclosure comprises a light chain variable region (VL) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL comprising the amino acid sequence of SEQ ID NO: 18.

In some embodiments, the anti-TfR1 antibody of the present disclosure comprises a VH comprising an amino acid sequence that is at least 75% (e.g., 75%, 80%, 85%, 90%, 95%, 98%, or 99%) identical in the framework regions to the VH comprising the amino acid sequence of SEQ ID NO: 17. Alternatively or in addition (e.g., in addition), in some embodiments, the anti-TfR1 antibody of the present disclosure comprises a VL comprising an amino acid sequence that is at least 75% (e.g., 75%, 80%, 85%, 90%, 95%, 98%, or 99%) identical in the framework regions to the VL comprising the amino acid sequence of SEQ ID NO: 18.

In some embodiments, the anti-TfR1 antibody of the present disclosure comprises a VH comprising the amino acid sequence of SEQ ID NO: 17. Alternatively or in addition (e.g., in addition), in some embodiments, the anti-TfR1 antibody of the present disclosure comprises a VL comprising the amino acid sequence of SEQ ID NO: 18.

In some embodiments, the anti-TfR1 antibody of the present disclosure comprises a heavy chain comprising an amino acid sequence least 75% (e.g., 75%, 80%, 85%, 90%, 95%, 98%, or 99%) identical to the amino acid sequence of SEQ ID NO: 19. In some embodiments, the anti-TfR1 antibody of the present disclosure is a Fab that comprises a heavy chain comprising an amino acid sequence least 75% (e.g., 75%, 80%, 85%, 90%, 95%, 98%, or 99%) identical to the amino acid sequence of SEQ ID NO: 19. Alternatively or in addition (e.g., in addition), the anti-TfR1 antibody of the present disclosure comprises a light chain comprising an amino acid sequence least 75% (e.g., 75%, 80%, 85%, 90%, 95%, 98%, or 99%) identical to the amino acid sequence of SEQ ID NO: 20. Alternatively or in addition (e.g., in addition), the anti-TfR1 antibody of the present disclosure is a Fab that comprises a light chain comprising an amino acid sequence least 75% (e.g., 75%, 80%, 85%, 90%, 95%, 98%, or 99%) identical to the amino acid sequence of SEQ ID NO: 20.

In some embodiments, the anti-TfR1 antibody of the present disclosure comprises a heavy chain comprising the amino acid sequence of SEQ ID NO: 19. In some embodiments, the anti-TfR1 antibody of the present disclosure is a Fab that comprises a heavy chain comprising the amino acid sequence of SEQ ID NO: 19. Alternatively or in addition (e.g., in addition), the anti-TfR1 antibody of the present disclosure comprises a light chain comprising the amino acid sequence of SEQ ID NO: 20. Alternatively or in addition (e.g., in addition), the anti-TfR1 antibody of the present disclosure is a Fab that comprises a light chain comprising the amino acid sequence of SEQ ID NO: 20.

In some embodiments, the anti-TfR1 antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. As such, it should be appreciated that an antibody specified as having a sequence comprising an N-terminal glutamate or glutamine residue encompasses antibodies that have undergone pyroglutamate formation resulting from a post-translational modification. In some embodiments, pyroglutamate formation occurs in a heavy chain sequence. In some embodiments, pyroglutamate formation occurs in a light chain sequence.

### Oligonucleotides

In some embodiments, an oligonucleotide of the complexes described herein is a single stranded oligonucleotide. In some embodiments, the oligonucleotide is useful for targeting DMD (e.g., for exon skipping). In some embodiments, an oligonucleotide that is useful for targeting DMD (e.g., for exon skipping) targets a DMD allele (e.g., a mutated DMD allele). In some embodiments, an oligonucleotide useful for targeting DMD (e.g., for exon skipping) targets a region of a DMD RNA (e.g., the Dp427m transcript of SEQ ID NO: 24). In some embodiments, an oligonucleotide useful for targeting DMD (e.g., for exon skipping) comprises a region of complementarity to a DMD RNA (e.g., the Dp427m transcript of SEQ ID NO: 23). In some embodiments, an oligonucleotide useful for targeting DMD (e.g., for exon skipping) comprises a region of complementarity to an exon (e.g., exons 8, 23, 43, 44, 45, 46, 50, 51, 52, 53, or 55) or an intron of a DMD RNA. In some embodiments, an oligonucleotide useful for targeting DMD (e.g., for exon skipping) targets a splicing donor site, a splicing acceptor site, a branch point, or an exonic splicing enhancer (ESE) of a DMD RNA (e.g., a DMD pre-mRNA encoded by Homo sapiens DMD gene (e.g., NCBI Accession No. NG_012232.1). In some embodiments, an oligonucleotide useful for targeting DMD (e.g., for exon skipping) targets an exonic splicing enhancer (ESE) sequence in DMD (e.g., an ESE sequence of exon 23, 44, 45, 46, 50, 51, 52, 53, or 55).

Examples of DMD RNA sequences and exon sequences that may be targeted by an oligonucleotide of a complex are provided below.

Homo sapiens dystrophin (DMD), transcript variant Dp427m, mRNA (NCBI Reference Sequence: NM_004006.2) (SEQ ID NO: 23).

Homo sapiens dystrophin (DMD), transcript variant Dp427m, exon 51
(nucleotide positions 7554-7786 of NCBI Reference Sequence: NM_004006.2)
(SEQ ID NO: 24)
CTCCTACTCAGACTGTTACTCTGGTGACACAACCTGTGGTTACTAAGGAAACTGCC
ATCTCCAAACTAGAAATGCCATCTTCCTTGATGTTGGAGGTACCTGCTCTGGCAGA
TTTCAACCGGGCTTGGACAGAACTTACCGACTGGCTTTCTCTGCTTGATCAAGTTA
TAAAATCACAGAGGGTGATGGTGGGTGACCTTGAGGATATCAACGAGATGATCAT
CAAGCAGAAG
Homo sapiens dystrophin (DMD), transcript variant Dp427m, exon 8
(nucleotide positions 894-1075 of NCBI Reference Sequence: NM_004006.2)
(SEQ ID NO: 25)
ATGTTGATACCACCTATCCAGATAAGAAGTCCATCTTAATGTACATCACATCACTC
TTCCAAGTTTTGCCTCAACAAGTGAGCATTGAAGCCATCCAGGAAGTGGAAATGTT
GCCAAGGCCACCTAAAGTGACTAAAGAAGAACATTTTCAGTTACATCATCAAATG
CACTATTCTCAACAG
Homo sapiens dystrophin (DMD), transcript variant Dp427m, exon 23
(nucleotide positions 3194-3406 of NCBI Reference Sequence: NM_004006.2)
(SEQ ID NO: 26)
GCTTTACAAAGTTCTCTGCAAGAGCAACAAAGTGGCCTATACTATCTCAGCACCAC
TGTGAAAGAGATGTCGAAGAAAGCGCCCTCTGAAATTAGCCGGAAATATCAATCA
GAATTTGAAGAAATTGAGGGACGCTGGAAGAAGCTCTCCTCCCAGCTGGTTGAGC
ATTGTCAAAAGCTAGAGGAGCAAATGAATAAACTCCGAAAAATTCAG
Homo sapiens dystrophin (DMD), transcript variant Dp427m, exon 43
(nucleotide positions 6362-6534 of NCBI Reference Sequence: NM_004006.2)
(SEQ ID NO: 27)
AATATAAAAGATAGTCTACAACAAAGCTCAGGTCGGATTGACATTATTCATAGCA
AGAAGACAGCAGCATTGCAAAGTGCAACGCCTGTGGAAAGGGTGAAGCTACAGG
AAGCTCTCTCCCAGCTTGATTTCCAATGGGAAAAAGTTAACAAAATGTACAAGGA
CCGACAAGG
Homo sapiens dystrophin (DMD), transcript variant Dp427m, exon 44
(nucleotide positions 6535-6682 of NCBI Reference Sequence: NM_004006.2)
(SEQ ID NO: 28)
GCGATTTGACAGATCTGTTGAGAAATGGCGGCGTTTTCATTATGATATAAAGATAT
TTAATCAGTGGCTAACAGAAGCTGAACAGTTTCTCAGAAAGACACAAATTCCTGA
GAATTGGGAACATGCTAAATACAAATGGTATCTTAAG
Homo sapiens dystrophin (DMD), transcript variant Dp427m, exon 45
(nucleotide positions 6683-6858 of NCBI Reference Sequence: NM_004006.2)
(SEQ ID NO: 36)
GAACTCCAGGATGGCATTGGGCAGCGGCAAACTGTTGTCAGAACATTGAATGCAA
CTGGGGAAGAAATAATTCAGCAATCCTCAAAAACAGATGCCAGTATTCTACAGGA
AAAATTGGGAAGCCTGAATCTGCGGTGGCAGGAGGTCTGCAAACAGCTGTCAGAC
AGAAAAAAGAG
Homo sapiens dystrophin (DMD), transcript variant Dp427m, exon 46
(nucleotide positions 6859-7006 of NCBI Reference Sequence: NM_004006.2)
(SEQ ID NO: 29)
GCTAGAAGAACAAAAGAATATCTTGTCAGAATTTCAAAGAGATTTAAATGAATTT
GTTTTATGGTTGGAGGAAGCAGATAACATTGCTAGTATCCCACTTGAACCTGGAAA
AGAGCAGCAACTAAAAGAAAAGCTTGAGCAAGTCAAG
Homo sapiens dystrophin (DMD), transcript variant Dp427m, exon 50
(nucleotide positions 7445-7553 of NCBI Reference Sequence: NM_004006.2)
(SEQ ID NO: 30)
AGGAAGTTAGAAGATCTGAGCTCTGAGTGGAAGGCGGTAAACCGTTTACTTCAAG
AGCTGAGGGCAAAGCAGCCTGACCTAGCTCCTGGACTGACCACTATTGGAGCCT
Homo sapiens dystrophin (DMD), transcript variant Dp427m, exon 51
(nucleotide positions 7554-7786 of NCBI Reference Sequence: NM_004006.2)
(SEQ ID NO: 31)
CTCCTACTCAGACTGTTACTCTGGTGACACAACCTGTGGTTACTAAGGAAACTGCC
ATCTCCAAACTAGAAATGCCATCTTCCTTGATGTTGGAGGTACCTGCTCTGGCAGA
TTTCAACCGGGCTTGGACAGAACTTACCGACTGGCTTTCTCTGCTTGATCAAGTTA
TAAAATCACAGAGGGTGATGGTGGGTGACCTTGAGGATATCAACGAGATGATCAT
CAAGCAGAAG
Homo sapiens dystrophin (DMD), transcript variant Dp427m, exon 52
(nucleotide positions 7787-7904 of NCBI Reference Sequence: NM_004006.2)
(SEQ ID NO: 32)
GCAACAATGCAGGATTTGGAACAGAGGCGTCCCCAGTTGGAAGAACTCATTACCG
CTGCCCAAAATTTGAAAAACAAGACCAGCAATCAAGAGGCTAGAACAATCATTAC
GGATCGAA
Homo sapiens dystrophin (DMD), transcript variant Dp427m, exon 53
(nucleotide positions 7905-8116 of NCBI Reference Sequence: NM_004006.2)
(SEQ ID NO: 33)
TTGAAAGAATTCAGAATCAGTGGGATGAAGTACAAGAACACCTTCAGAACCGGAG
GCAACAGTTGAATGAAATGTTAAAGGATTCAACACAATGGCTGGAAGCTAAGGAA
GAAGCTGAGCAGGTCTTAGGACAGGCCAGAGCCAAGCTTGAGTCATGGAAGGAG
GGTCCCTATACAGTAGATGCAATCCAAAAGAAAATCACAGAAACCAAG
Homo sapiens dystrophin (DMD), transcript variant Dp427m, exon 55
(nucleotide positions 8272-8461 of NCBI Reference Sequence: NM_004006.2)
(SEQ ID NO: 34)
GGTGAGTGAGCGAGAGGCTGCTTTGGAAGAAACTCATAGATTACTGCAACAGTTC
CCCCTGGACCTGGAAAAGTTTCTTGCCTGGCTTACAGAAGCTGAAACAACTGCCA
ATGTCCTACAGGATGCTACCCGTAAGGAAAGGCTCCTAGAAGACTCCAAGGGAGT
AAAAGAGCTGATGAAACAATGGCAA

In some embodiments, an oligonucleotide useful for targeting DMD (e.g., for exon skipping) is 15-40 (e.g., 15-40, 15-35, 15-30, 15-25, 15-20, 20-40, 20-35, 20-30, 20-25, 25-40, 25-35, 25-30, 25-28, 28-30, 30-40, 30-32, 32-35, 30-35, or 35-40) nucleotides in length. In some embodiments, an oligonucleotide useful for targeting DMD (e.g., for exon skipping) is 15, 16, 17, 18, 19, 20, 21, 22, 23, 24, 25, 26, 27, 28, 29, or 30 nucleotides in length, optionally 20-35, or 30 nucleotides in length.

In some embodiments, an oligonucleotide useful for targeting DMD (e.g., for exon skipping) comprises a region of complementarity of at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides to a DMD RNA. In some embodiments, an oligonucleotide useful for targeting DMD (e.g., for exon skipping) comprises a region of complementarity of at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides to an exon of a DMD RNA.

In some embodiments, an oligonucleotide useful for targeting DMD (e.g., for exon skipping) comprises a region of complementarity of at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides to a DMD sequence as set forth in any one of SEQ ID NOs: 23-34.

In some embodiments, an oligonucleotide useful for targeting DMD (e.g., for exon skipping) comprises a region of complementarity of at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides to a target sequence as set forth in SEQ ID NO: 22 (CTAGAAATGCCATCTTCCTTGATGTTGGAG). In some embodiments, an oligonucleotide useful for targeting DMD (e.g., for exon skipping) comprises at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides of a sequence as set forth in SEQ ID NO: 21

(CTCCAACATCAAGGAAGATGGCATTTCTAG).

In some embodiments, an oligonucleotide useful for targeting DMD (e.g., for exon skipping) comprises the nucleotide sequence of SEQ ID NO: 21. In some embodiments, any one of the oligonucleotides provided herein is a PMO.

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, any one or more of the thymine bases (T's) in any one of the oligonucleotides provided herein (e.g., the oligonucleotide as set forth in SEQ ID NO: 21) may independently and optionally be uracil bases (U's), and/or any one or more of the U's in the oligonucleotides provided herein may independently and optionally be T's.

### Compositions

In some embodiments, compositions described herein comprise complexes (i.e., a plurality of complexes), each of which complex comprises an antibody (e.g., anti-TFR1 antibody) covalently linked to one or more oligonucleotides (e.g., an oligonucleotide described herein), wherein the antibody comprises a heavy chain comprising a heavy chain variable region (VH) and a heavy chain constant region, and a light chain comprising a light chain variable region (VL) and a light chain constant region. In some embodiments, the antibody of such complexes comprises a CDR-H1, CDR-H2, CDR-H3, CDR-L1, CDR-L2, and CDR-L3 as set forth in Table 2. Complexes of a composition described herein can comprise any structure provided herein, e.g., a structure of formula (I) (e.g., comprising a group of the formula (Ia), formula (Ib), formula (Ic), or formula (Id)) or formula (A).

In some embodiments, compositions described herein comprise complexes (i.e., a plurality of complexes) wherein each complex comprises a structure of formula (I): [R¹]ₙ₁—R², in which each R¹ independently comprises a compound comprising an oligonucleotide (e.g., an oligonucleotide described herein) and is covalently linked to R², wherein R² comprises an antibody (e.g., anti-TfR1 antibody) comprising a heavy chain comprising a heavy chain variable region (VH) and a heavy chain constant region, and a light chain comprising a light chain variable region (VL) and a light chain constant region. In some embodiments, each R¹ of a complex is independently covalently linked to a different amino acid residue (e.g., lysine or cysteine) of R².

In some embodiments, the value of n1 of complexes in the composition is independently and optionally an integer from one up to the number of amino acid residues to which conjugation is desired or targeted (e.g., the number of lysine residues) in the antibody (e.g., an antibody comprised within R²). In some embodiments, the value of n1 of each complex in the composition is independently and optionally selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, the value of n1 of each complex in the composition is independently and optionally selected from 1, 2, 3, 4, 5, 6, 7, 8, 9, 10, 11, 12, 13, 14, 15, 16, 17, 18, 19, 20, 21, 22, 23, 24, 25 and 26. In some embodiments, the value of n1 of each complex in the composition is independently selected and optionally from an integer in the range of 1 to 27, 1 to 26, 1 to 10, 1 to 5, or 1 to 3. In some embodiments, the average value of n1 of complexes of the composition is in the range of 1 to 2, 1 to 3, 1 to 5, 1 to 10, 1 to 26, or 1 to 27. In some embodiments, compositions described herein comprise complexes in which the value of n1 is 0. In some embodiments, the average value of n1 of complexes of the composition is in the range of 1 to 5 (e.g., 1-5, 1-4, 1-3, 1-2, 2-4, 3-5, 1-4.6, 1-4.5, 1-4.4, 1-4.3, 1-4.2, 1-3.5, 1-2.5, 1.1-5, 1.1-4.5, 1.1-4, 1.1-3.5, 1.1-3, 1.1-2.5, 1.1-2.2, 1.2-5, 1.2-4.5, 1.2-4, 1.2-3.5, 1.2-3, 1.2-2.5, 1.2-2.2, 1.3-5, 1.3-4.5, 1.3-4, 1.3-3.5, 1.3-3, 1.3-2.5, 1.3-2.2, 1.4-5, 1.4-4.5, 1.4-4, 1.4-3.5, 1.4-3, 1.4-2.5, 1.4-2.2, 1.5-5, 1.5-4.5, 1.5-4, 1.5-3.5, 1.5-3, 1.5-2.5, 1.5-2.2, 1.6-3, 1.6-2.5, 1.6-2.2, 1.7-3, 1.7-2.5, 1.7-2.2, 1.8-3, 1.8-2.5, or 1.8-2.2).

In some embodiments, a composition described herein comprises antibody that is not conjugated to an oligonucleotide (e.g., in trace amounts) and antibody conjugated to one or more oligonucleotides. In some embodiments, antibody that is not conjugated to an oligonucleotide may be referred to as a compound comprising a structure of formula (I): [R¹]ₙ₁—R², for which n1 is zero. Accordingly, in some embodiments, a composition for administration to a subject in the methods described herein comprises compounds (e.g., complexes) comprising a structure of formula (I): [R¹]ₙ₁—R², for which each R¹ independently comprises a group comprising an oligonucleotide, R² comprises an antibody and n1 is independently an integer of zero or greater that reflects the number of instances of R¹ in each compound (e.g., complex). In some embodiments, the fraction of compounds comprising a structure of formula (I): [R¹]ₙ₁—R², in a composition, for which n1 is zero, compared with all compounds of that structure in the composition for which n1 is one or greater, is less than 10%, less than 5%, less than 1% less than 0.5%, less than 0.1%, less than 0.05%, or less than 0.01%. As such, in some embodiments, the average value of n1 of complexes in a composition disclosed herein is in the range of 1 to 5 (e.g., 1-5, 1-4, 1-3, 1-2, 2-4, 3-5, 1-4.6, 1-4.5, 1-4.4, 1-4.3, 1-4.2, 1-3.5, 1-2.5, 1.1-5, 1.1-4.5, 1.1-4, 1.1-3.5, 1.1-3, 1.1-2.5, 1.1-2.2, 1.2-5, 1.2-4.5, 1.2-4, 1.2-3.5, 1.2-3, 1.2-2.5, 1.2-2.2, 1.3-5, 1.3-4.5, 1.3-4, 1.3-3.5, 1.3-3, 1.3-2.5, 1.3-2.2, 1.4-5, 1.4-4.5, 1.4-4, 1.4-3.5, 1.4-3, 1.4-2.5, 1.4-2.2, 1.5-5, 1.5-4.5, 1.5-4, 1.5-3.5, 1.5-3, 1.5-2.5, 1.5-2.2, 1.6-3, 1.6-2.5, 1.6-2.2, 1.7-3, 1.7-2.5, 1.7-2.2, 1.8-3, 1.8-2.5, or 1.8-2.2).

### Formulations

Complexes provided herein are formulated in a manner suitable for pharmaceutical use. In some embodiments, complexes can be delivered to a subject using a formulation that minimizes degradation, facilitates delivery and/or (e.g., and) uptake, or provides another beneficial property to complexes in the formulation. Accordingly, in some embodiments, it has been found that formulating complexes (e.g., complexes comprising a PMO covalently linked with a Fab) with histidine and/or sucrose is particularly advantageous for pharmaceutical use, e.g., as described herein. Thus, in some embodiments, provided herein are formulations (e.g., aqueous solutions, lyophilized forms) comprising complexes together with histidine and/or sucrose. In some embodiments, provided herein are formulations comprising complexes together with histidine and/or sucrose in frozen forms. In some embodiments, formulations described herein comprise complexes (e.g., a plurality of complexes comprising a PMO covalently linked with a Fab), histidine, and sucrose. In some embodiments, formulations comprising muscle-targeting complexes (e.g., complexes comprising a PMO covalently linked with a Fab) are formulated with histidine and/or sucrose in aqueous solutions. In some embodiments, formulation comprising a plurality of the complexes, histidine, and sucrose can be lyophilized (e.g., for storage). In some embodiments, the lyophilized formulation may be reconstituted (e.g., with water) for administration to a subject. Such formulations can be suitably prepared such that when administered to a subject, either into the immediate environment of a target cell or systemically, a sufficient amount of the complexes enter target muscle cells.

In some embodiments, formulations are provided herein that comprise complexes (i.e., a plurality of complexes), each of which complex comprises a phosphorodiamidate morpholino oligomer (PMO) covalently linked to an antibody. In some embodiments, provided herein is a formulation comprising complexes, in which each complex comprises a phosphorodiamidate morpholino oligomer (PMO) covalently linked to an anti-TfR1 antibody, optionally wherein the antibody of such complexes comprises a CDR-H1, CDR-H2, CDR-H3, CDR-L1, CDR-L2, and CDR-L3 as set forth in Table 2, and further, in some embodiments, wherein the complexes are formulated with histidine (e.g., L-histidine) and sucrose. In some embodiments, the antibody is an anti-TfR1 antibody.

In some embodiments, formulations are provided that comprise complexes of the formula: [R¹]ₙ₁—R², in which each R¹ independently comprises a compound comprising an oligonucleotide (e.g., a PMO) and R² comprises an antibody (e.g., anti-TfR1 antibody), and in which n1 is an integer of one or greater representing the number of instances of R¹ in the complex. In some embodiments, formulations are provided that comprise a plurality of complexes wherein each complex comprises a structure of formula (I): [R¹]ₙ₁—R², in which each R¹ independently comprises a compound comprising an oligonucleotide (e.g., a PMO) and R² comprises an antibody (e.g., anti-TfR1 antibody), and in which in each complex n1 is an integer independently of one or greater representing the number of instances of R¹ in each complex.

In some embodiments, formulations described herein comprise complexes comprising an antibody that comprises a CDR-H1, CDR-H2, CDR-H3, CDR-L1, CDR-L2, and CDR-L3 as set forth in Table 2. In some embodiments, the antibody comprises a VH comprising the amino acid sequence of SEQ ID NO: 17 and a VL comprising the amino acid sequence of SEQ ID NO: 18. In some embodiments, the antibody is a Fab and comprises a heavy chain comprising the amino acid sequence of SEQ ID NO: 19 and a light chain comprising the amino acid sequence of SEQ ID NO: 20.

In some embodiments, the value of n1 of each complex in the formulation is independently and optionally an integer from one up to the number of amino acid residues to which conjugation is desired or targeted (e.g., the number of lysine residues) in the antibody (R²). In some embodiments, the value of n1 of each complex in the formulation is independently and optionally selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, the value of n1 of each complex in the formulation is independently and optionally selected from 1, 2, 3, 4, 5, 6, 7, 8, 9, 10, 11, 12, 13, 14, 15, 16, 17, 18, 19, 20, 21, 22, 23, 24, 25 and 26. In some embodiments, the value of n1 of each complex in the formulation is independently selected and optionally from an integer in the range of 1 to 27, 1 to 26, 1 to 10, 1 to 5, or 1 to 3. In some embodiments, the average value of n1 of complexes of the formulation is in the range of 1 to 3, 1 to 5, 1 to 10, 1 to 26 or 1 to 27.

In some embodiments, a formulation described herein comprises antibody that is not conjugated to an oligonucleotide (e.g., in trace amounts) and antibody conjugated to one or more oligonucleotides. In some embodiments, antibody that is not conjugated to an oligonucleotide antibody may be referred to as a compound comprising a structure of formula (I): [R¹]ₙ₁—R², for which n1 is zero. Accordingly, in some embodiments, formulations are provided that comprise compounds (e.g., complexes) comprising a structure of formula (I): [R¹]ₙ₁—R², for which each R¹ independently comprises a group comprising an oligonucleotide, R² comprises an antibody and n1 is independently an integer of zero or greater that reflects the number of instances of R¹ in each compound (e.g., complex). In some embodiments, the fraction of compounds comprising a structure of formula (I): [R¹]ₙ₁—R², in a formulation, for which n1 is zero, compared with all compounds of that structure in the formulation for which n1 is one or greater, is less than 10%, less than 5%, less than 1% less than 0.5%, less than 0.1%, less than 0.05%, or less than 0.01%. In some embodiments, the average value of n1 of complexes of the formulation is in the range of 1 to 5 (e.g., 1-5, 1-4, 1-3, 1-2, 2-4, 3-5, 1-4.6, 1-4.5, 1-4.4, 1-4.3, 1-4.2, 1-3.5, 1-2.5, 1.1-5, 1.1-4.5, 1.1-4, 1.1-3.5, 1.1-3, 1.1-2.5, 1.1-2.2, 1.2-5, 1.2-4.5, 1.2-4, 1.2-3.5, 1.2-3, 1.2-2.5, 1.2-2.2, 1.3-5, 1.3-4.5, 1.3-4, 1.3-3.5, 1.3-3, 1.3-2.5, 1.3-2.2, 1.4-5, 1.4-4.5, 1.4-4, 1.4-3.5, 1.4-3, 1.4-2.5, 1.4-2.2, 1.5-5, 1.5-4.5, 1.5-4, 1.5-3.5, 1.5-3, 1.5-2.5, 1.5-2.2, 1.6-3, 1.6-2.5, 1.6-2.2, 1.7-3, 1.7-2.5, 1.7-2.2, 1.8-3, 1.8-2.5, or 1.8-2.2).

In some embodiments, each instance of R¹ in a complex herein (e.g., a complex of a formulation provided herein) is conjugated to a different amino acid residue of the antibody. In some embodiments, each different amino acid comprises an F-amino group (e.g., lysine, arginine). However, in some embodiments, each different amino acid to which R¹ is covalently linked is a cysteine. In some embodiments, R¹ is directly covalently linked to an amino acid residue of the antibody. However, in some embodiments, R¹ is indirectly covalently linked to an amino acid of the antibody, e.g., covalently linked to a glycosylation site on the amino acid. In some embodiments, formulations are provided in which complexes for which R¹ is covalently linked to an amino acid residue residing in a CDR region of the antibody are present in only trace amounts, or in undetectable amount, or not at all. In some embodiments, formulations are provided in which complexes for which R¹ is covalently linked to an amino acid residue residing in a CDR region of the antibody are not detectable in the formulation using standard detection techniques.

In some embodiments, formulations provided herein comprise complexes that comprise a structure of formula (I): [R¹]ₙ₁—R², in which each instance of R¹ in a complex of a formulation provided herein independently comprises a group of the formula (Ia):

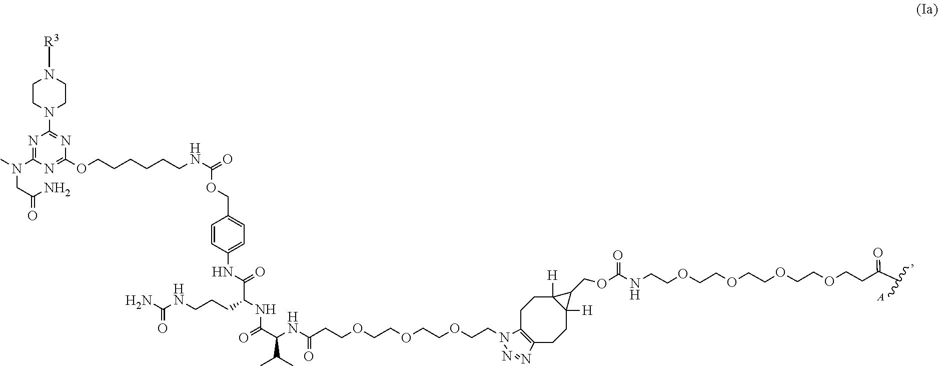

in which R³ comprises an oligonucleotide, e.g., a phosphorodiamidate morpholino oligomer (PMO); and R¹ is covalently linked (e.g., indirectly or directly linked, e.g., directly linked) to R² at attachment point A. In some embodiments, in each complex n1 is independently an integer (e.g., of one or greater) representing the number of instances of R¹ in each complex, In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R² comprises an antibody that is a Fab fragment. In some embodiments, R³ comprises an oligonucleotide, e.g., a phosphorodiamidate morpholino oligomer (PMO) comprising the base sequence of CTCCAACATCAAGGAAGATGGCATTTCTAG (SEQ ID NO: 21). In some embodiments, R² comprises an antibody a Fab and each R¹ is covalently linked at attachment point A to a different amino acid residue of the antibody Fab, optionally wherein each different amino acid residue is a lysine. In some embodiments, in each complex n1 is independently an integer (e.g., an integer in the range of 1-27, 1-26, 1-10, 1-5, or 1-3). In some embodiments, formulations provided herein comprise complexes that comprise a structure of formula (I): [R¹]ₙ₁—R², wherein n1 is 0. In some embodiments, the average value of n1 of complexes of the composition is in the range of 1 to 5 (e.g., 1-5, 1-4, 1-3, 1-2, 2-4, 3-5, 1-4.6, 1-4.5, 1-4.4, 1-4.3, 1-4.2, 1-3.5, 1-2.5, 1.1-5, 1.1-4.5, 1.1-4, 1.1-3.5, 1.1-3, 1.1-2.5, 1.1-2.2, 1.2-5, 1.2-4.5, 1.2-4, 1.2-3.5, 1.2-3, 1.2-2.5, 1.2-2.2, 1.3-5, 1.3-4.5, 1.3-4, 1.3-3.5, 1.3-3, 1.3-2.5, 1.3-2.2, 1.4-5, 1.4-4.5, 1.4-4, 1.4-3.5, 1.4-3, 1.4-2.5, 1.4-2.2, 1.5-5, 1.5-4.5, 1.5-4, 1.5-3.5, 1.5-3, 1.5-2.5, 1.5-2.2, 1.6-3, 1.6-2.5, 1.6-2.2, 1.7-3, 1.7-2.5, 1.7-2.2, 1.8-3, 1.8-2.5, or 1.8-2.2).

In some embodiments, formulations provided herein comprise complexes that comprise a structure of formula (I): [R¹]ₙ₁—R², in which each instance of R¹ in a complex of a formulation provided herein comprises a group of the formula (Ib):

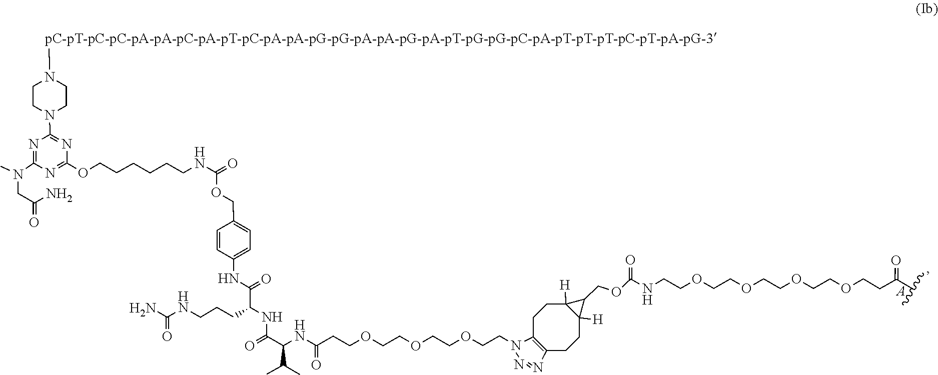

in which -pN indicates a base position of a phosphorodiamidate morpholino oligomer (PMO); R¹ is covalently linked (e.g., indirectly or directly linked, e.g., directly linked) to R² at attachment point A, wherein -p reflects a phosphorodiamidate linkage, and wherein N corresponds to a nucleobase of adenine (A), cytosine (C), guanine (G), or thymine (T), such that the PMO comprises a base sequence of CTCCAACATCAAGGAAGATGGCATTTCTAG (SEQ ID NO: 21). In some embodiments, in each complex n1 is independently an integer (e.g., of one or greater) representing the number of instances of R¹ in each complex, and each R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, R² comprises a Fab and each R¹ is covalently linked at attachment point A to a different amino acid residue of the Fab, optionally wherein each different amino acid residue is a lysine. In some embodiments, formulations provided herein comprise complexes that comprise a structure of formula (I): [R¹]ₙ₁—R², wherein n1 is 0. In some embodiments, the average value of n1 of complexes of the composition is in the range of 1 to 5 (e.g., 1-5, 1-4, 1-3, 1-2, 2-4, 3-5, 1-4.6, 1-4.5, 1-4.4, 1-4.3, 1-4.2, 1-3.5, 1-2.5, 1.1-5, 1.1-4.5, 1.1-4, 1.1-3.5, 1.1-3, 1.1-2.5, 1.1-2.2, 1.2-5, 1.2-4.5, 1.2-4, 1.2-3.5, 1.2-3, 1.2-2.5, 1.2-2.2, 1.3-5, 1.3-4.5, 1.3-4, 1.3-3.5, 1.3-3, 1.3-2.5, 1.3-2.2, 1.4-5, 1.4-4.5, 1.4-4, 1.4-3.5, 1.4-3, 1.4-2.5, 1.4-2.2, 1.5-5, 1.5-4.5, 1.5-4, 1.5-3.5, 1.5-3, 1.5-2.5, 1.5-2.2, 1.6-3, 1.6-2.5, 1.6-2.2, 1.7-3, 1.7-2.5, 1.7-2.2, 1.8-3, 1.8-2.5, or 1.8-2.2).

In some embodiments, formulations provided herein comprise complexes that comprise a structure of formula (I): [R¹]ₙ₁—R², in which each instance of R¹ in a complex of a formulation provided herein comprises a group of the formula (Ic):

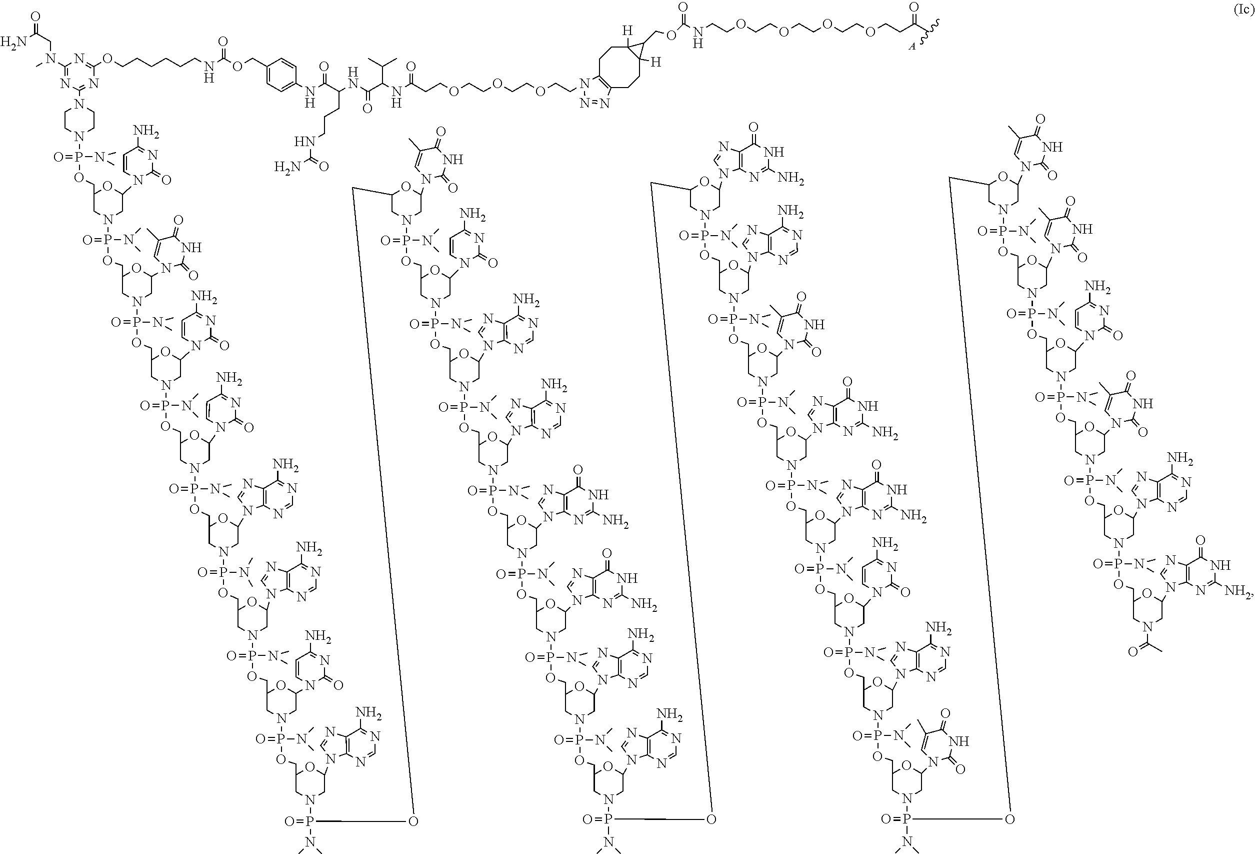

wherein R¹ is covalently linked (e.g., indirectly or directly linked, e.g. directly linked) to R² at attachment point A.

In some embodiments, formulations provided herein comprise complexes that comprise a structure of formula (I: [R¹]ₙ₁—R², in which each instance of R¹ in a complex of a formulation provided herein is:

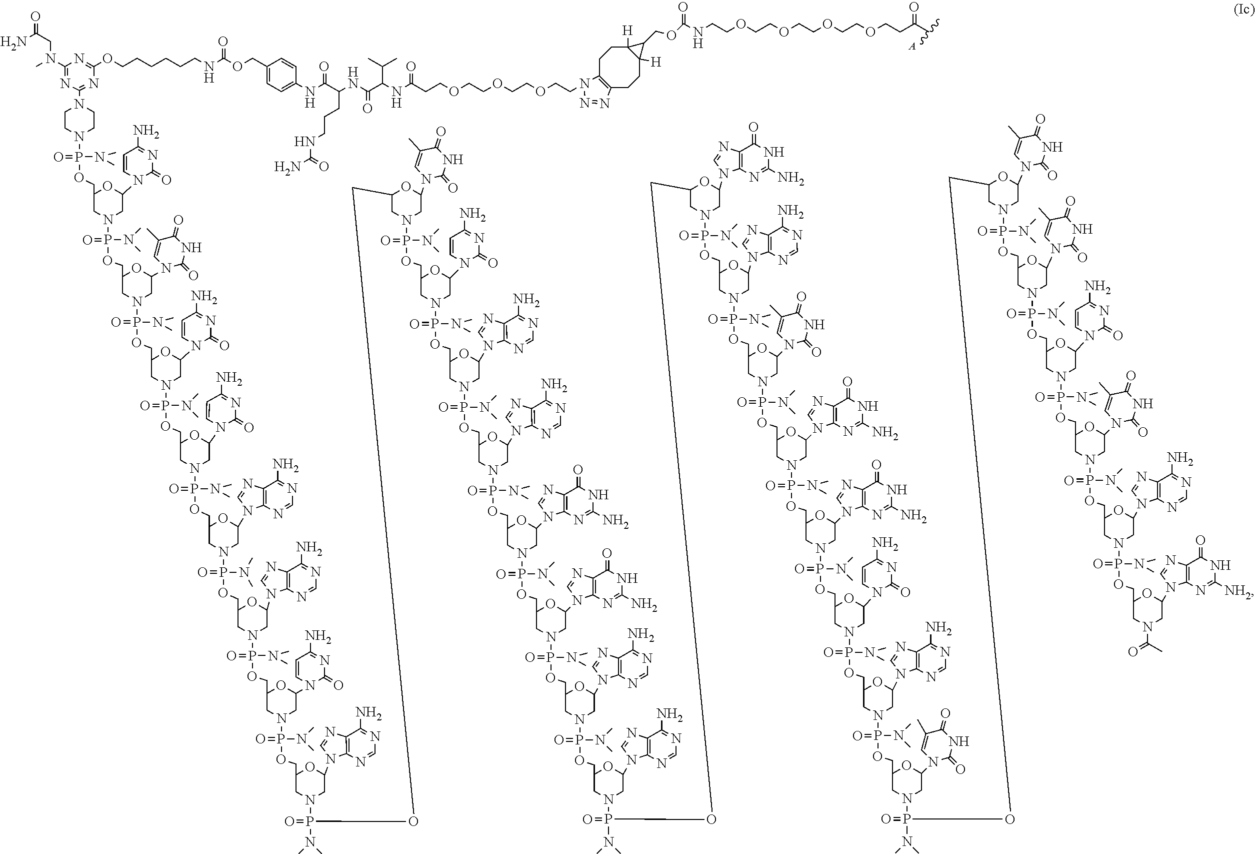

wherein R¹ is covalently linked (e.g., indirectly or directly linked, e.g. directly linked) to R² at attachment point A. In some embodiments, in each complex n1 is independently an integer (e.g., of one or greater) representing the number of instances of R¹ in each complex. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, R² comprises a Fab and each R¹ is covalently linked at attachment point A to a different amino acid residue of the Fab, optionally wherein each different amino acid residue is a lysine. In some embodiments, formulations described herein further comprise complexes that comprise a structure of formula (I): [R¹]ₙ₁—R², wherein n1 is 0. In some embodiments, the average value of n1 of complexes of the composition is in the range of 1 to 5 (e.g., 1-5, 1-4, 1-3, 1-2, 2-4, 3-5, 1-4.6, 1-4.5, 1-4.4, 1-4.3, 1-4.2, 1-3.5, 1-2.5, 1.1-5, 1.1-4.5, 1.1-4, 1.1-3.5, 1.1-3, 1.1-2.5, 1.1-2.2, 1.2-5, 1.2-4.5, 1.2-4, 1.2-3.5, 1.2-3, 1.2-2.5, 1.2-2.2, 1.3-5, 1.3-4.5, 1.3-4, 1.3-3.5, 1.3-3, 1.3-2.5, 1.3-2.2, 1.4-5, 1.4-4.5, 1.4-4, 1.4-3.5, 1.4-3, 1.4-2.5, 1.4-2.2, 1.5-5, 1.5-4.5, 1.5-4, 1.5-3.5, 1.5-3, 1.5-2.5, 1.5-2.2, 1.6-3, 1.6-2.5, 1.6-2.2, 1.7-3, 1.7-2.5, 1.7-2.2, 1.8-3, 1.8-2.5, or 1.8-2.2).

In some embodiments, formulations provided herein comprise complexes that comprise a structure of formula (Id):

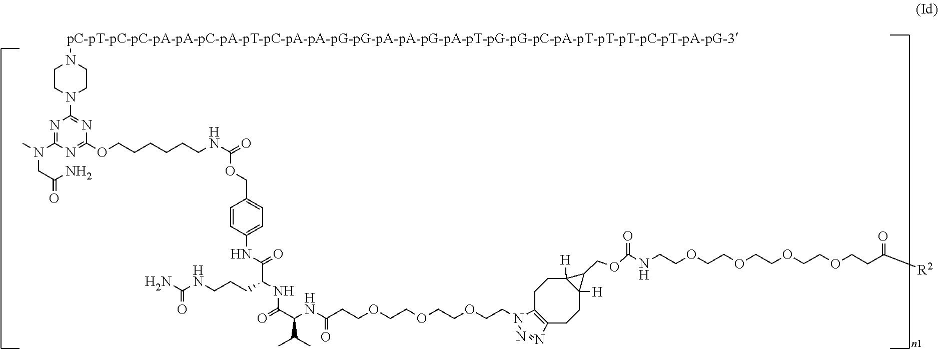

in which -pN indicates a base position of a phosphorodiamidate morpholino oligomer (PMO); wherein -p reflects a phosphorodiamidate linkage, and wherein N corresponds to a nucleobase of adenine (A), cytosine (C), guanine (G), or thymine (T), such that the PMO comprises a base sequence of CTCCAACATCAAGGAAGATGGCATTTCTAG (SEQ ID NO: 21); wherein R² comprises an antibody comprising a sequence as set forth in Table 2; wherein in each complex n1 is independently an integer (e.g., of one or greater) representing the number of instances of the group enclosed by square brackets, wherein each instance of the group enclosed by square brackets is covalently linked to a different amino acid residue of the antibody (e.g., Fab), optionally wherein each different amino acid residue is a lysine. In some embodiments, R² comprises an antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody (e.g., a Fab) comprising a VH comprising the amino acid sequence of SEQ ID NO: 17 and/or comprising a VL comprising the amino acid sequence of SEQ ID NO: 18. In some embodiments, R² comprises an antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody (e.g., a Fab) comprising a heavy chain comprising the amino acid sequence of SEQ ID NO: 19 and/or comprising a light chain comprising the amino acid sequence of SEQ ID NO: 20. In some embodiments, in each complex n1 is independently an integer (e.g., an integer in the range of 1-27, 1-26, 1-10, 1-5, or 1-3). In some embodiments, R² comprises an antibody (e.g., a Fab) that is covalently linked via different amino acid residue of the antibody (e.g., Fab), optionally wherein each different amino acid residue is a lysine. In some embodiments, formulations described herein further comprise complexes in which n1 is 0. In some embodiments, the average value of n1 of complexes of the composition is in the range of 1 to 5 (e.g., 1-5, 1-4, 1-3, 1-2, 2-4, 3-5, 1-4.6, 1-4.5, 1-4.4, 1-4.3, 1-4.2, 1-3.5, 1-2.5, 1.1-5, 1.1-4.5, 1.1-4, 1.1-3.5, 1.1-3, 1.1-2.5, 1.1-2.2, 1.2-5, 1.2-4.5, 1.2-4, 1.2-3.5, 1.2-3, 1.2-2.5, 1.2-2.2, 1.3-5, 1.3-4.5, 1.3-4, 1.3-3.5, 1.3-3, 1.3-2.5, 1.3-2.2, 1.4-5, 1.4-4.5, 1.4-4, 1.4-3.5, 1.4-3, 1.4-2.5, 1.4-2.2, 1.5-5, 1.5-4.5, 1.5-4, 1.5-3.5, 1.5-3, 1.5-2.5, 1.5-2.2, 1.6-3, 1.6-2.5, 1.6-2.2, 1.7-3, 1.7-2.5, 1.7-2.2, 1.8-3, 1.8-2.5, or 1.8-2.2).

In some embodiments, complexes provided in the formulations described herein comprise a structure of formula (A):

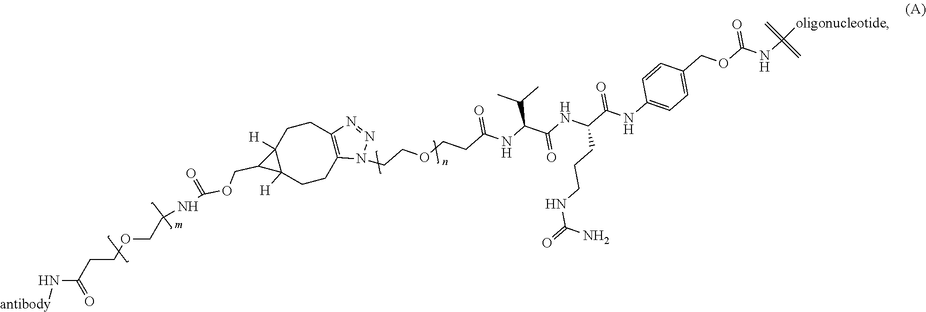

wherein y is 0-15 (e.g., 3) and z is 0-15 (e.g., 4). In some embodiments, the antibody is an anti-TfR1 antibody (e.g., the anti-TfR1 antibody provided in Table 2). In some embodiments, the oligonucleotide is a PMO and comprises the base sequence of SEQ ID NO: 21. In some embodiments, the amide shown adjacent to the anti-TfR1 antibody in the structure (A) results from a reaction with an amine of the antibody, such as a lysine epsilon amine. In some embodiments, a complex described herein comprises an anti-TfR1 Fab covalently linked via a lysine of the Fab to the 5′ end of a PMO. In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)2 fragment, an scFv, or an Fv.

In some embodiments, provided is a formulation comprising complexes described herein wherein a concentration of the complexes in the formulation therein is between 1-50 mg/mL of the complex, optionally 10-50 mg/ml or 20-35 mg/mL (e.g., 1-10 mg/mL, 10-15 mg/mL, 15-20 mg/mL, 20-22 mg/mL, 22-24 mg/ml, 24-26 mg/ml, 24-25 mg/ml, 25-26 mg/ml, 22-25 mg/mL, 25-27 mg/mL, 27-29 mg/mL, 29-30 mg/mL, 25-30 mg/mL, 29-31 mg/ml, 30-31 mg/ml, 31-32 mg/ml, 30-32 mg/mL, 32-33 mg/ml, 32-35 mg/mL, 30-35 mg/mL, 35-40 mg/mL, 40-45 mg/mL, 45-50 mg/mL), optionally approximately 25 mg/mL (e.g., 25 mg/mL) or approximately 30 mg/mL (e.g., 30 mg/mL).

In some embodiments, any one or a plurality of the complexes described herein is formulated with the histidine (e.g., L-histidine) and the sucrose in a lyophilized form (e.g., lyophilized powder).

In some embodiments, any one or a plurality of the complexes described herein is formulated with the histidine (e.g., L-histidine) and the sucrose in an aqueous solution. In some embodiments, the histidine (e.g., L-histidine) is present in the aqueous solution at a concentration in the range of 10-50 mM, 10-20 mM, 20 mM to 30 mM, or 20 mM to 40 mM, e.g., 20-22 mM, 22-24 mM, 24-25 mM, 25-26 mM, 24-26 mM, 26-27 mM, 24-27 mM, 27-28 mM, 28-29 mM, 29-30 mM, 27-30 mM, approximately 22-27 mM, approximately 23-26 mM, approximately 24-26 mM, approximately 26-28 mM, approximately 28-30 mM, approximately 30-32 mM, approximately 32-35 mM, approximately 35-40 mM, 40-45 mM, 45-50 mM, approximately 25 mM, or optionally, 25 mM. In some embodiments, the sucrose is present in the aqueous solution at a concentration in the range of 5% to 15% weight per volume (w/v %), for example, 8-15% w/v %, 9-15% w/v %, 9-11% w/v %, 9.5-11% w/v %, or for example, in the range of 5-6 w/v %, 6-7 w/v %, 7-8 w/v %, 8-9 w/v %, 9-10 w/v %, 10-11 w/v %, 11-12% w/v %, 10-12 w/v %, 12-13% w/v %, 13-14% w/v %, 12-14 w/v %, 14-15 w/v %, or 8-12 w/v %. In some embodiments, the sucrose is present in the aqueous solution at a concentration in the range of 8-12 w/v % (e.g., 10 w/v %). In some embodiments, the aqueous solution has a pH in the range of 5.0 to 7.0, for example, 5.0-5.2, 5.2-5.4, 5.4-5.6, 5.6-5.8, 5.8-6.0, 5.9-6.0, 5.9-6.1, 6.0-6.1; for example, 5.5 to 6.5, or for example, in the pH range of 5.5-5.8, 5.8-6.0, 5.9-6.1, 6.0-6.1, 6.0-6.2, 6.2-6.4, 6.4-6.5, 6.5-6.7, 6.7-6.8, 6.8-6.9, 6.9-7.0, 7.0-7.1, or 5.8-6.2. In some embodiments, the aqueous solution has a pH in the range of 5.8-6.2 (e.g., 5.8-6.0, 5.8-6.1, 5.9-6.1). In some embodiments, the aqueous solution has a pH in the range of 5.9-6.2. In some embodiments, the aqueous solution has a pH in the range of 6.0-6.1 (e.g., about 6.0, or 6.0).

In some embodiments, provided is a formulation (e.g., in aqueous solution) described herein comprising one or a plurality of complexes, histidine, and sucrose, In some embodiments, any one of the formulations described herein is an aqueous solution, wherein the histidine (e.g., L-histidine) is present in the aqueous solution at a concentration of 25 mM, wherein the sucrose is present in the aqueous solution at a concentration of 10 w/v %, and wherein the aqueous solution is at a pH of about 6.0 (e.g., 6.0, 5.9-6.1).

In some embodiments, provided is a formulation (e.g., in aqueous solution) described herein comprising a plurality of complexes, histidine, and sucrose, wherein the histidine (e.g., L-histidine) is present in the aqueous solution at a concentration of 25 mM, wherein the sucrose is present in the aqueous solution at a concentration of 10 w/v %, and wherein the pH of about 6.0 (e.g., 6.0, 5.9-6.1), and the concentration of complexes in the formulation is 10-50 mg/ml or 20-35 mg/mL (e.g., 1-10 mg/mL, 10-15 mg/mL, 15-20 mg/mL, 20-22 mg/mL, 22-24 mg/ml, 24-26 mg/ml, 22-25 mg/mL, 25-27 mg/mL, 27-29 mg/mL, 29-31 mg/ml, 29-30 mg/mL, 30-31 mg/ml, 31-32 mg/ml, 25-30 mg/mL, 30-32 mg/mL, 32-35 mg/mL, 30-35 mg/mL, 35-40 mg/mL, 40-45 mg/mL, 45-50 mg/mL), optionally 25 mg/mL or 30 mg/mL.

As described herein, in some embodiments, formulations provided herein comprise sucrose. In some embodiments, sucrose serves at least in part as a lyoprotectant. In some embodiments, the sucrose is from a plant, e.g., grass, fruit, or vegetable (e.g., root vegetable) source (e.g., beet (e.g., sugar beet, for example, Saccharum spp.)), sugarcane (e.g., Beta vulgaris), dates, sugar maple, sweet sorghum, apples, oranges, carrots, molasses, maple syrup, corn sweeteners) or animal product (e.g., honey). In some embodiments, the sucrose is from beet or sugarcane (e.g., beet sucrose, sugarcane sucrose). In some embodiments, a lyoprotectant other than sucrose may be used, e.g., trehalose, mannitol, lactose, polyethylene glycol, or polyvinyl pyrrolidone. However, in some embodiments, a collapse temperature modifier (e.g., dextran, ficoll, or gelatin) may be provided in a formulation.

In some embodiments, any one or a plurality of the complexes described herein is formulated with histidine and sucrose in a lyophilized form (e.g., lyophilized powder). In some embodiments, the lyophilized form (e.g., lyophilized powder) is obtained by lyophilization of any one of the aqueous solutions described herein.

In some embodiments, provided is a product (e.g., lyophilized formulation described herein), produced by a process comprising lyophilizing an aqueous solution of a formulation (e.g., in aqueous form) described herein.

In some embodiments, any one or a plurality of the complexes described herein is formulated with histidine and sucrose in a frozen form (e.g., a frozen aqueous solid). In some embodiments, the frozen form (e.g., frozen aqueous solid) is obtained by freezing of any one of the aqueous solutions described herein. A frozen form may be frozen to a temperature of less than −20° C. (e.g., less than −20° C., less than −30° C., less than −40° C., less than −50° C., less than −60° C., less than −70° C., less than −80° C., or lower).

In some embodiments, provided is a product (e.g., frozen formulation described herein), produced by a process comprising freezing an aqueous solution of a formulation (e.g., in aqueous form) described herein.

In some embodiments, a pharmaceutical composition is formulated to be compatible with its intended route of administration. Examples of routes of administration include parenteral, e.g., intravenous, intradermal, subcutaneous, administration. Typically, the route of administration is intravenous or subcutaneous.

### Methods of Use/Treatment

Complexes comprising an anti-TfR1 antibody (e.g., Fab) covalently linked to a molecular payload (e.g., oligonucleotide, e.g., phosphorodiamidate morpholino oligomer (PMO)) as described herein are effective in treating a subject having a dystrophinopathy, e.g., Duchenne Muscular Dystrophy. In some embodiments, complexes comprise a molecular payload that is an oligonucleotide, e.g., an antisense oligonucleotide that facilitates exon skipping of an mRNA expressed from a mutated DMD allele.

In some embodiments, a subject may be a human subject, a non-human primate subject, a rodent subject, or any suitable mammalian subject. In some embodiments, the non-human primate subject is a cynomolgus monkey. In some embodiments, the subject is human. In some embodiments, the subject is a human subject that is between 2-60 (e.g., 2-60, 2-50, 2-40, 2-30, 2-20, 2-10) years of age. In some embodiments, the subject is a human subject that is between 5-30 (e.g., 5, 6, 7, 8, 9, 10, 11, 12, 13, 14, 15, 16, 17, 18, 19, 20, 21, 22, 23, 24, 25, 26, 27, 28, 29, or 30) years old. In some embodiments, the subject is a human subject that is between 5-12 (e.g., 5, 6, 7, 8, 9, 10, 11, or 12) years of age. In some embodiments, the subject is a human subject that is between 4-16 (e.g., 4-16, 5-16, 6-16, 7-16, 8-16, 9-16, 10-16, 11-16, 12-16, 13-16, 14-16, 15-16, 4-15, 5-15, 6-15, 7-15, 8-15, 9-15, 10-15, 11-15, 12-15, 13-15, 14-15, 4-14, 5-14, 6-14, 7-14, 8-14, 9-14, 10-14, 11-14, 12-14, 13-14, 4-13, 5-13, 6-13, 7-13, 8-13, 9-13, 10-13, 11-13, 12-13, 4-12, 5-12, 6-12, 7-12, 8-12, 9-12, 10-12, 11-12, 4-11, 5-11, 6-11, 7-11, 8-11, 9-16, 10-11, 4-10, 5-10, 6-10, 7-10, 8-10, 9-10, 4-9, 5-9, 6-9, 7-9, 8-9, 4-9, 5-9, 6-9, 7-9, 8-9, 4-8, 5-8, 6-8, 7-8, 4-7, 5-7, 6-7, 4-6, 5-6, or 4-5) years of age. In some embodiments, the subject is a human subject that is about 4, 5, 6, 7, 8, 9, 10, 11, 12, 13, 14, 15, or 16 years of age.

In some embodiments, a subject may have Duchenne muscular dystrophy or other dystrophinopathy. In some embodiments, a subject has a mutated DMD allele, which may optionally comprise at least one mutation in a DMD exon that causes a frameshift mutation and leads to improper RNA splicing/processing. In some embodiments, a subject is suffering from symptoms of a severe dystrophinopathy, e.g., muscle atrophy or muscle loss. In some embodiments, a subject has an asymptomatic increase in serum concentration of creatine phosphokinase (CK) and/or (e.g., and) muscle cramps with myoglobinuria. In some embodiments, a subject has a progressive muscle disease, such as Duchenne or Becker muscular dystrophy or DMD-associated dilated cardiomyopathy (DCM). In some embodiments, a subject is not suffering from symptoms of a dystrophinopathy. In some embodiments, a subject is ambulant. In some embodiments, a subject is non-ambulant.

In some embodiments, a subject has a mutation in a DMD gene that is amenable to exon 51 skipping. In some embodiments, a complex as described herein is effective in treating a subject having a mutation in a DMD gene that is amenable to exon 51 skipping. In some embodiments, a complex comprises an oligonucleotide, e.g., an oligonucleotide that facilitates skipping of exon 51 of a pre-mRNA, such as in a pre-mRNA encoded from a mutated DMD gene (e.g., a mutated DMD gene that is amenable to exon 51 skipping).

An aspect of the disclosure includes methods involving administering to a subject a formulation comprising an effective amount of complex(es) as described herein. In some embodiments, an effective amount of a pharmaceutical composition that comprises complex(es) comprising an antibody (e.g., Fab) described herein covalently linked to an oligonucleotide (e.g., PMO) described herein can be administered to a subject in need of treatment. In some embodiments, a pharmaceutical composition is administered systemically. In some embodiments, a pharmaceutical composition comprising complex(es) as described herein may be administered by a suitable route, which may include intravenous administration, e.g., as a bolus or by continuous infusion over a period of time. In some embodiments, administration may be performed by intravenous, intramuscular, intraperitoneal, intracerebrospinal, subcutaneous, intra-articular, intrasynovial, or intrathecal routes. In some embodiments, a pharmaceutical composition comprising complex(es) as described herein is administered by infusion (e.g., intravenous infusion). In some embodiments, a pharmaceutical composition comprising a plurality of complexes described herein may be in solid form, aqueous form, or a liquid form. In some embodiments, an aqueous or liquid form may be nebulized or lyophilized. In some embodiments, a lyophilized form may be reconstituted with an aqueous or liquid solution.

In some embodiments, provided are methods of and/or uses for treating a subject having a mutated DMD allele associated with Duchenne Muscular Dystrophy, comprising administering to the subject a formulation comprising a complex or plurality of complexes described herein with an effective amount of the complex(es). In some embodiments, provided are methods of and/or uses for promoting the expression or activity of a dystrophin protein in a subject, the methods comprising contacting the cell with the formulation comprising a plurality of complexes described herein with an effective amount of the complex(es). In some embodiments, the dystrophin protein is a truncated dystrophin protein. The truncated dystrophin protein is functional (e.g., retains activities of a wild-type dystrophin protein). In some embodiments, the truncated dystrophin protein retains partial function of a wild-type dystrophin protein. In some embodiments, the method comprises administering a lyophilized form (e.g., lyophilized powder) of a formulation comprising a plurality of complexes described herein, comprising reconstituting a lyophilized form of the formulation in an aqueous solution, and administering the aqueous solution of the formulation to a subject in need thereof. For example, in some embodiments, a lyophilized form of the formulation comprising a complex or plurality of complexes is shipped and/or stored in the lyophilized form, reconstituted at a location for administering the aqueous solution of the formulation (e.g., healthcare provider location), and administered in the reconstituted form (e.g., as an aqueous solution) by injection or intravenously, e.g., by infusion. In some embodiments, the subject has a mutated DMD allele comprises a mutation amenable to exon 51 skipping. In some embodiments, the mutated DMD allele comprises a frameshift mutation in exon 51.

In some embodiments, a pharmaceutical composition is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising an anti-TfR1 antibody (e.g., a Fab) covalently linked to a molecular payload (e.g., oligonucleotide, e.g., phosphorodiamidate morpholino oligomer (PMO)) is administered at an effective concentration that confers therapeutic effect on a subject. Effective amounts vary, as recognized by those skilled in the art, depending on the severity of the disease, unique characteristics of the subject being treated, e.g. age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g. the half-life of the complex(es) in a subject, generally will contribute to determination of the concentration of pharmaceutical composition that is used for treatment. The frequency of administration may be empirically determined and adjusted to maximize the efficacy of the treatment. The efficacy of treatment may be assessed using any suitable methods. In some embodiments, the efficacy of treatment may be assessed by evaluation or observation of symptoms associated with a dystrophinopathy, e.g. muscle atrophy or muscle weakness, through measures of a subject's self-reported outcomes, e.g. mobility, self-care, usual activities, pain/discomfort, and anxiety/depression, or by quality-of-life indicators, e.g. lifespan. In some embodiments, a pharmaceutical composition that comprises a complex comprising a muscle-targeting agent covalently linked to a molecular payload described herein is administered to a subject at an effective concentration sufficient to modulate activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

### EXAMPLES

### Example 1. Exon-Skipping Activity of Anti-TfR1 Conjugates in Duchenne Muscular Dystrophy Patient Myotubes

This study evaluated the exon-skipping activities of anti-TfR1 conjugates comprising the anti-TfR1 Fab having the VH and VL sequences shown in Table 2 covalently linked (through lysine conjugation) via a linker comprising a Valine-Citrulline sequence to a DMD exon 51-skipping antisense oligonucleotide (ASO). The DMD exon 51-skipping ASO is a PMO and comprises the nucleotide sequence of SEQ ID NO: 21. The conjugates comprise a structure of:

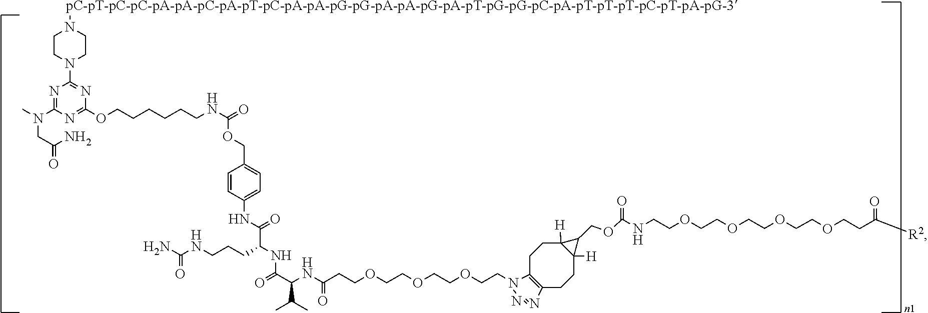

wherein R² is the anti-TfR1 Fab shown in Table 2, and wherein in each conjugate n1 is independently an integer of zero or greater.

Immortalized human myoblasts bearing an exon 52 deletion were thawed and seeded at a density of 1×10⁶ cell/flask in Promocell Skeletal Cell Growth Media (with 5% FBS and 1× Pen-Strep) and allowed to grow to confluency. Once confluent, cells were trypsinized and pelleted via centrifugation and resuspended in fresh Promocell Skeletal Cell Growth Media. The cell number was counted and cells were seeded into Matrigel-coated 96-well plates at a density of 50 k cells/well. Cells were allowed to recover for 24 hours. Cells were induced to differentiate by aspirating the growth media and replacing with differentiation media with no serum. Cells were then treated with the DMD exon 51-skipping oligonucleotide (not covalently linked to an antibody—“naked”) at a concentration of 10 μM oligonucleotide or treated with the conjugate to a final concentration of 10 μM oligonucleotide equivalent. Cells were incubated with test articles for ten days then total RNA was harvested from the 96 well plates. cDNA synthesis was performed on 75 ng of total RNA, and mutation specific PCRs were performed to evaluate the degree of exon 51 skipping in each cell type. Mutation-specific PCR products were run on a 4% agarose gel and visualized using SYBR gold. Densitometry was used to calculate the relative amounts of the skipped and unskipped amplicon and exon skipping was determined as a ratio of the Exon 51 skipped amplicon divided by the total amount of amplicon present:

$$\% Exon Skipping = \frac{Skipped Amplicon}{( Skipped Amplicon + Unskipped Amplicon )} * 100$$

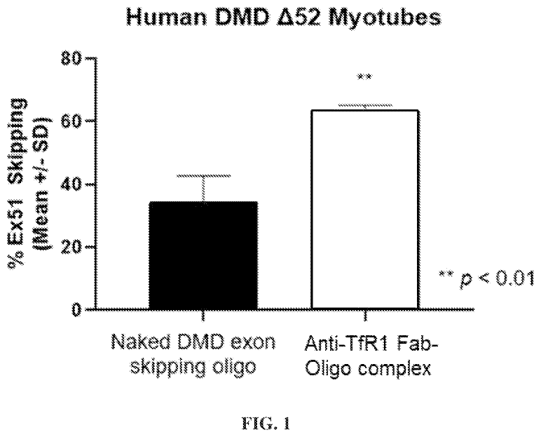

The results demonstrate that the conjugates resulted in enhanced exon skipping compared to the same DMD exon 51-skipping oligonucleotide that is not covalently linked to an antibody in patient myotubes (FIG. 1). This indicates that an anti-TfR1 Fab (e.g., having a sequence set forth in Table 2) facilitated cellular internalization of the conjugate into muscle cells resulting in activity of the exon 51-skipping oligonucleotide in the muscle cells. Similarly, an anti-TfR1 antibody can facilitate internalization of a conjugate comprising the anti-TfR1 antibody covalently linked to other exon skipping oligonucleotides (e.g., an exon skipping oligonucleotide provided herein, such as an exon 51 skipping oligonucleotide) into muscle cells and facilitate activity of the exon skipping oligonucleotide in the muscle cells.

### Example 2. Exon Skipping Activity of Anti-TfR1 Fab-ASO Conjugate In Vivo in Cynomolgus Monkeys

The anti-TfR1 oligonucleotide conjugates described in Example 1 were tested for their exon skipping activity in vivo in healthy non-human primates. Naïve male cynomolgus monkeys (n=4-5 per group) were administered two doses of vehicle, 30 mg/kg naked ASO (i.e., not covalently linked to an antibody), or 122 mg/kg anti-TfR1 Fab covalently linked to the DMD exon 51-skipping oligonucleotide (30 mg/kg ASO equivalent) via intravenous infusion on days 1 and 8. Animals were sacrificed and tissues harvested either 2 weeks or 4 weeks after the first dose was administered. Total RNA was collected from tissue samples using a Promega Maxwell® RSC instrument and cDNA synthesis was performed using qScript cDNA SuperMix. Assessment of exon 51 skipping was performed using end-point PCR.

Capillary electrophoresis of the PCR products was used to assess exon skipping, and % exon 51 skipping was calculated using the following formula:

$$\% Exon Skipping = \frac{Molarity of Skipped Band}{Molarity of Skipped Band + Molarity of Unskipped Band} * 100.$$

Calculated exon 51 skipping results are shown in Table 4.

TABLE 4
Exon 51 skipping of DMD in cynomolgus monkey DMD

| | Time | | | | |
|---|---|---|---|---|---|
| | 2 weeks | | | 4 weeks | |
| | | ASO | | ASO | |
| Group | Vehicle | alone^{a} | Conjugate | alone^{a} | Conjugate |
| Conjugate dose^{b} | 0 | n/a | 122 | n/a | 122 |
| ASO alone Dose^{c} | 0 | 30 | 30 | 30 | 30 |
| Quadriceps ^{d} | 0.00 | 1.216 | 4.906 | 0.840 | 1.708 |
| | (0.00) | (1.083) | (3.131) | (1.169) | (1.395) |
| Diaphragm ^{d} | 0.00 | 1.891 | 7.315 | 0.717 | 9.225 |
| | (0.00) | (2.911) | (1.532) | (1.315) | (4.696) |
| Heart ^{d} | 0.00 | 0.043 | 3.42 | 0.00 | 4.525 |
| | (0.00) | (0.096) | (1.192) | (0.00) | (1.400) |
| Biceps ^{d} | 0.00 | 0.607 | 3.129 | 1.214 | 4.863 |
| | (0.00) | (0.615) | (0.912) | (1.441) | (3.881) |
| Tibialis anterior ^{d} | 0.00 | 0.699 | 1.042 | 0.384 | 0.816 |
| | (0.00) | (0.997) | (0.685) | (0.615) | (0.915) |
| Gastrocnemius ^{d} | 0.00 | 0.388 | 2.424 | 0.00 | 5.393 |
| | (0.00) | (0.573) | (2.329) | (0.00) | (2.695) |

^{a}ASO = antisense oligonucleotide.
^{b}Conjugate doses are listed as mg/kg of anti-TfR1 Fab-ASO conjugate.
^{c}ASO doses are listed as mg/kg ASO equivalent of the anti-TfR1 Fab-ASO dose.
^{d} Exon skipping values are mean % exon 51 skipping with standard deviations (n = 5) in parentheses.

Tissue ASO accumulation was also quantified using a hybridization ELISA with a probe complementary to the ASO sequence. A standard curve was generated and ASO levels (in ng/g) were derived from a linear regression of the standard curve. The ASO was distributed to all tissues evaluated at a higher level following the administration of the anti-TfR1 Fab-ASO conjugate as compared to the administration of unconjugated ASO (not covalently linked to antibody). Intravenous administration of unconjugated ASO resulted in levels of ASO that were close to background levels in all tissues evaluated at 2 and 4 weeks after the first dose was administered. Administration of the conjugate resulted in distribution of ASO through the tissues evaluated with a rank order of heart>diaphragm>bicep>quadriceps>gastrocnemious>tibialis anterior 2 weeks after first dosing. The duration of tissue concentration was also assessed. ASO levels were detectable at 4 weeks post dose in all tissues (Table 5). This indicates that the anti-TfR1 Fab shown in Table 2 enabled cellular internalization of the conjugate into muscle cells in vivo, resulting in activity of the exon skipping oligonucleotide in the muscle cells.

TABLE 5
Tissue distribution of DMD exon 51
skipping ASO in cynomolgus monkeys

| | Time | | | | |
|---|---|---|---|---|---|
| | 2 weeks | | | 4 weeks | |
| | | ASO | Conju- | ASO | Conju- |
| Group | Vehicle | alone^{a} | gate | alone^{a} | gate |
| Conjugate Dose^{b} | 0 | n/a | 122 | n/a | 122 |
| ASO alone Dose^{c} | 0 | 30 | 30 | 30 | 30 |
| Quadriceps ^{d} | 0 | 696.8 | 2436 | 197 | 682 |
| | (59.05) | (868.15) | (954.0) | (134) | (281) |
| Diaphragm ^{d} | 0± | 580.02 | 6750 | 60 | 3131 |
| | (144.3) | (360.11) | (2256) | (120) | (1618) |
| Heart ^{d} | 0 | 1449 | 27138 | 943 | 30410 |
| | (396.03) | (1337) | (6315) | (1803) | (9247) |
| Biceps ^{d} | 0 | 615.63 | 2840 | 130 | 1326 |
| | (69.58) | (335.17) | (980.31) | (80) | (623) |
| Tibialis anterior ^{d} | 0 | 564.71 | 1591 | 169 | 1087 |
| | (76.31) | (327.88) | (253.50) | (110) | (514) |
| Gastrocnemius ^{d} | 0 | 705.47 | 2096 | 170 | 1265 |
| | (41.15) | (863.75) | (474.04) | (69) | (272) |

^{a}ASO = Antisense oligonucleotide.
^{b}Conjugate doses are listed as mg/kg of anti-TfR1 Fab-ASO conjugate.
^{c}ASO doses are listed as mg/kg ASO or ASO equivalent of the anti-TfR1 Fab-ASO conjugate dose.
^{d} ASO values are mean concentrations of ASO in tissue as ng/g with standard deviations (n = 5) in parentheses.

### Example 3. Comparison of Freeze-Thaw Stability Tests of Different Exemplary Formulations

The following formulations were prepared. The formulation comprises the anti-TfR1 Fab having the VH/VL sequences shown in Table 2 covalently linked (through lysine conjugation) via a linker comprising a Valine-Citrulline sequence to a DMD exon 51-skipping ASO, wherein the anti-TfR1 Fab-ASO conjugate was at a concentration of 25 mg/ml, in a 2 mL glass vial with 500 μL fill volume. The DMD exon 51-skipping ASO is a PMO and comprises the nucleotide sequence of SEQ ID NO: 21.
Formulation 1: 25 mM histidine, 10% sucrose, pH 6
Formulation 2: 25 mM histidine, 10% sucrose, pH 5.5
Formulation 3: 25 mM histidine, 10% sucrose, pH 6.5
Formulation 4: 25 mM histidine, 10% sucrose, 0.02% PS-80, pH 6
Formulation 5: 10 mM histidine, 10% sucrose, pH 6
Formulation 6: 50 mM histidine, 10% sucrose, pH 6
Formulation 7: 25 mM histidine, 150 mM NaCl, pH 6
Formulation 8: 25 mM phosphate, 150 mM NaCl, pH 7

The formulations were frozen at −80° C. and followed by thawing at ambient temperature (e.g., room temperature of approximately 20° C.), with 5×F/T (freeze/thaw) cycles, and held at 2-8° C. for 3-4 hours before analysis.

Formulation buffers prepared: The formulation drug substance (formulation with conjugate described herein) was buffer-exchanged with appropriate formulation buffer. Sartorius Vivaspin Filters were used (30 kDa MWCO). After 5 buffer changes, the anti-TfR1 Fab-ASO was concentrated to 25 mg/ml. The drug products were sterile filtered and loaded into the vials (a 700 μL fill was used for the vials). The sample formulations were visually observed at the following timepoints: T0 (week 0), T1 (week 1), T2 (week 2), T4 (week 4), T8 (week 8), and at the temperatures of −20° C., 2-8° C., 25° C., and 40° C.

At time points T0, T1, T2, T4, and T8, formulations 1-6 were observed to be clear and colorless, at all temperatures including −20° C., 2-8° C., 25° C., and 40° C. Formulations 7 and 8 were observed to be show precipitation with opalescence and particles, at all temperatures including −20° C., 2-8° C., 25° C., and 40° C.

### Example 4. Analysis of Thermal Stability of Exemplary Formulation

The following formulation was prepared. The formulation comprises the anti-TfR1 Fab having the VH/VL sequences shown in Table 2 having the VH/VL sequences shown in Table 2 covalently linked (through lysine conjugation) via a linker comprising a Valine-Citrulline sequence to a DMD exon 51-skipping ASO (PMO comprising the nucleotide sequence of SEQ ID NO: 21), wherein the anti-TfR1 Fab-ASO conjugate was at a concentration of 25 mg/ml, in a 2 mL glass vial with 500 μL fill volume. In this formulation (Formulation 1) were: 25 mM histidine, 10% sucrose, pH 6.

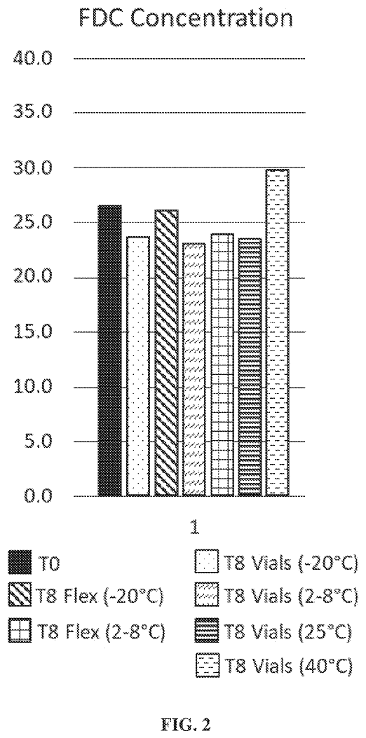

Formulation buffers were prepared: The formulation drug substance (formulation with conjugate described herein) was buffer-exchanged with appropriate formulation buffer. Sartorius Vivaspin Filters were used (30 kDa MWCO). After 5 buffer changes, the anti-TfR1 Fab-ASO was concentrated to 25 mg/ml. The drug products were sterile filtered and loaded into the vials (a 700 μL fill was used for the vials). Next, an analysis of the thermal stability of the formulation described above in Example 4 (“Formulation 1”) was conducted. This analysis shows the conjugate concentration of the anti-TfR1 Fab-ASO conjugate at the specified timepoints of T0 (week 0) and T8 (week 8)), and at the specified temperatures. See FIG. 2. Standard BCA (Bicinchoninic Acid) analysis was used to measure the protein conjugate concentration. BCA assay was carried out using standard procedure:
1) Prepare Working Reagent (WR). 100 μL of WR is required for each sample in the microplate procedure. Prepare WR by mixing 50:1 (BCA reagent A: BCA reagent B) thoroughly. 10 mL of WR reagent is needed for a full 96 well plate. Therefore, combine 10 mL of Reagent A with 200 μL of Reagent B and ensure buffer is at RT and used within 90 minutes of mixing. It is recommended to use freshly made working solution.
2) Prepare standard curve and dilution curve for unknown samples. 10 μL of standard or unknown sample is added to 200 μL of WR and incubated at 60° C. for 10 minutes. This is added to the first row of the plate and serial diluted 100 μL:100 μL in subsequent columns, 96-well clear plate: Standard: Known Fab: 15G11 (11 mg/mL). Prepare an 8-point standard curve and unknown sample dilution curve by doing 1:1 dilution for each point.
3) Incubate for 5 minutes.
4) Measure the absorbance at 480 nM on a plate reader.
5) Use standard curve to determine the protein concentration of each unknown sample (6 dilutions were measured).

In Formulation 1, slight decreases in concentration of the conjugate were observed in different containers (2 mL glass vial with 600 μL fill volume referred to as “vial”; plastic Flexboy bag with a 1 mL fill volume referred to as “flex”), indicating overall stability of this formulation at specified temperatures and in the specified containers. Less of a conjugate concentration decrease from week 0 (TO) to the timepoint at week 8 (T8) was observed for formulations in the plastic Flexboy bag as compared to the glass vial for temperatures −20° C. and 2-8° C. However, at timepoint 8 (T8) for Formulation 1 in the glass vials at 40° C., no decrease in conjugate concentration was observed.

### Example 5. Analysis of Material Adhesion to Container Types

A study was conducted to analyze material losses through adhesion of formulations, to evaluate possible adhesion to various plastics, comprising the exemplary anti-TfR1 Fab having the VH/VL sequences shown in Table 2 covalently linked (through lysine conjugation) via a linker comprising a Valine-Citrulline sequence to a DMD exon 51-skipping ASO (PMO comprising the nucleotide sequence of SEQ ID NO: 21). This TfR1 Fab-ASO conjugate formulated in Formulation 1 (25 mM histidine, 10% sucrose buffer (pH 6)) described in Example 3: showed less material loss. The study was conducted as follows: repeat the previous study of Example 3 at 25 mg/mL; add the formulations to the plastics and perform 1 freeze/thaw (F/T) cycle (freeze at −80° C. overnight, thaw at 2-8° C. for 4 hours); and analyze by standard tests, including Visual, BCA, SEC-HPLC (SEC). Following one freeze-thaw cycle at −80° C. as discussed, the concentration of the conjugate in Formulation 1 was measured. Standard BCA (Bicinchoninic Acid) analysis was used to measure the protein conjugate concentration, as disclosed in Example 4.

SEC-HPLC analytical method was as follows:
HPLC System Thermo Ultimate-3000 UHPLC system
Sample Concentration: 1 mg/mL (diluted with HPLC-grade water); Injection Volume: 10 μL
Buffer: 100 mM sodium phosphate, 100 mM NaCl, 15% Acetonitrile, pH 7.0
Run: Isocratic, 0.25 mL/min, 20 min
Column oven temperature: Ambient. Column: Waters AQUITY UPLC Protein BEH SEC Column 200 Å, 1.7 μm, 4.6×300 mm (P/N: 186005226)
Wavelengths: 280 nm and 260 nm Samples injected in duplicate, average values reported.

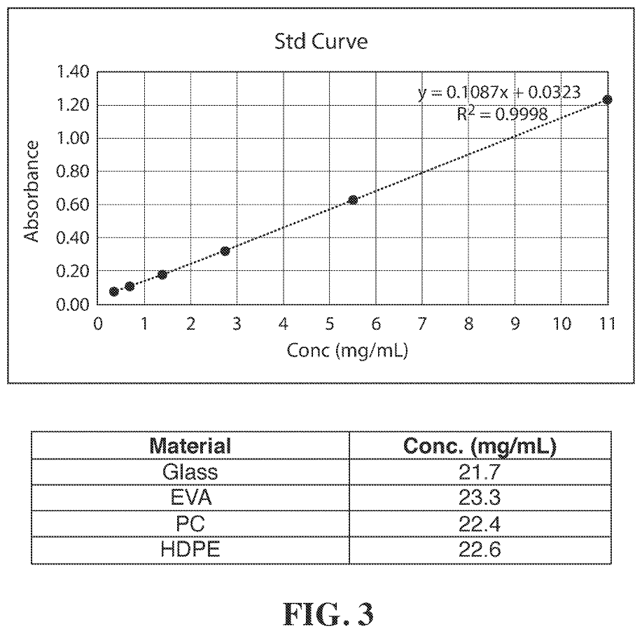

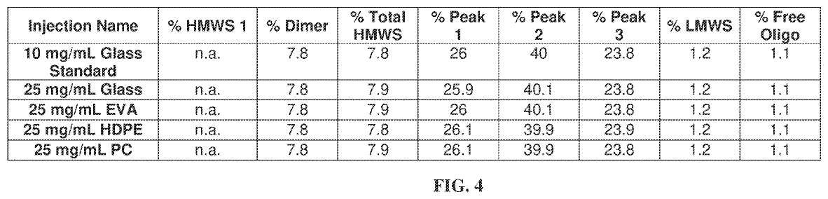

The strong stability and lack of surface adhesion to different plastic container types (ethylene vinyl acetate plastic (EVA), polycarbonates plastic (PC), High Density Poly Ethylene plastic (HDPE)) using the 25 mM histidine, 10% sucrose, pH 6.0 formulation of the conjugate is demonstrated collectively in FIGS. 3 and 4. FIGS. 3 and 4 show the good stability and lack of surface adhesion, demonstrated by a higher concentration of the conjugate in Formulation 1 for the plastic container types, as compared to the lower concentration of the conjugate for the glass container. FIG. 4 shows that via the SEC analysis, there were no significant changes in the composition of the HPLC peaks across all of the samples, including the sample with the 10 mg/ml glass standard (FDC conjugate which was at −10 mg/mL, was stored in glass, and was subjected to one freeze thaw cycle (frozen from shipping and held at 2-8° C.)), and the 25 mg/ml samples in glass or specified plastics (25 mg/mL conjugate concentrated in spin filters, then added to the indicated plastics or glass and subjected to one F/T cycle).

The results of FIG. 4 indicate good stability of Formulation 1.

### ADDITIONAL EMBODIMENTS

1. A formulation comprising complexes that comprise a phosphorodiamidate morpholino oligomer (PMO) covalently linked to an anti-transferrin receptor 1 (TfR1) antibody, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, and wherein the complexes are formulated with histidine and sucrose.
2. A formulation comprising complexes of the formula: [R¹]ₙ₁—R², wherein
each R¹ independently comprises a group of the formula:

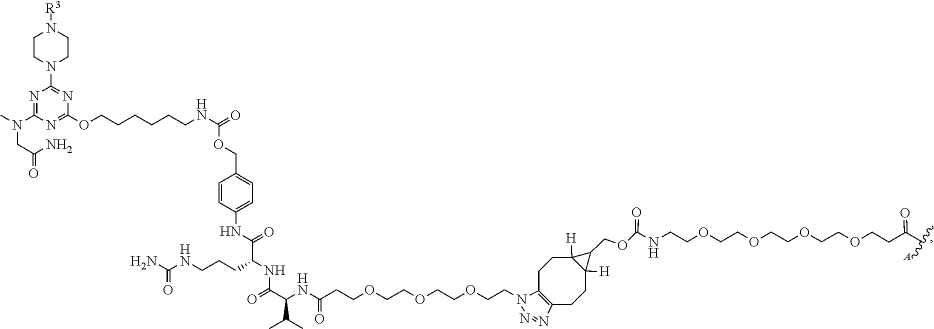

R² comprises an antibody, and
R³ is a phosphorodiamidate morpholino oligomer (PMO);
wherein R¹ is covalently linked to R² at attachment point A; and
wherein n1 is an integer of one or greater representing the number of instances of R¹, wherein each instance of R¹ is covalently linked to a different amino acid residue of the antibody, optionally wherein each different amino acid residue is a lysine;
wherein the complexes are formulated with histidine and sucrose,
optionally wherein the antibody is an anti-TfR1 antibody, and optionally wherein the average value of n1 of complexes in the formulation is in the range of 1 to 5.
3. The formulation of embodiment 2, wherein the antibody comprises:
a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16.
4. The formulation of any one of embodiments 1 to 3, wherein the formulation is in a lyophilized form.
5. The formulation of any one of embodiments 1 to 3, wherein the formulation is in an aqueous solution.
6. The formulation of embodiment 5, wherein the histidine is present in the aqueous solution at a concentration in the range of 10 mM to 50 mM.
7. The formulation of embodiment 5 or 6, wherein the sucrose is present in the aqueous solution at a concentration in the range of 5% to 15% weight per volume (w/v %).
8. The formulation of any one of embodiments 5 to 7, wherein the aqueous solution has a pH in the range of 5.0 to 7.0.
9. The formulation of any one of embodiments 5-8, wherein the histidine is present in the aqueous solution at a concentration of 25 mM and/or the sucrose is present in the aqueous solution at a concentration of 10 w/v % and/or the aqueous solution is at a pH of 6.0.
10. The formulation of any one of embodiments 1-9, wherein the antibody is a Fab fragment, a full-length IgG, a Fab′ fragment, a F(ab′)₂ fragment, an scFv, or an Fv.
11. The formulation of embodiment 10, wherein the antibody is a Fab fragment.
12. The formulation of any one of embodiments 1-11, wherein the antibody comprises a heavy chain variable region (VH) comprising an amino acid sequence at least 85% identical to SEQ ID NO: 17; and/or wherein the antibody comprises a light chain variable region (VL) comprising an amino acid sequence at least 85% identical to SEQ ID NO: 18, optionally wherein the antibody comprises a VH comprising the amino acid sequence of SEQ ID NO: 17 and a VL comprising the amino acid sequence of SEQ ID NO: 18.
13. The formulation of any one of embodiments 1-12, wherein the antibody comprises a heavy chain comprising an amino acid sequence at least 85% identical to SEQ ID NO: 19; and/or wherein the antibody comprises a light chain comprising an amino acid sequence at least 85% identical to SEQ ID NO: 20, optionally wherein the antibody comprises a heavy chain comprising the amino acid sequence of SEQ ID NO: 19 and a light chain comprising the amino acid sequence of SEQ ID NO: 20.
14. The formulation of any one of embodiments 1-13, wherein the PMO comprises a nucleotide sequence that is 15-35 nucleotides in length.
15. The formulation of any one of embodiments 1-14, wherein the PMO comprises a nucleotide sequence having a region of complementarity of at least 8 consecutive nucleotides in length to SEQ ID NO: 23, to SEQ ID NO: 24, or to SEQ ID NO: 22.
16. The formulation of any one of embodiments 1-15, wherein the PMO comprises at least 8 consecutive nucleotides of a nucleotide sequence as set forth in SEQ ID NO: 21, optionally wherein the PMO comprises the nucleotide sequence of SEQ ID NO: 21.
17. The formulation of any one of embodiments 2-16, wherein each R¹ comprises formula:

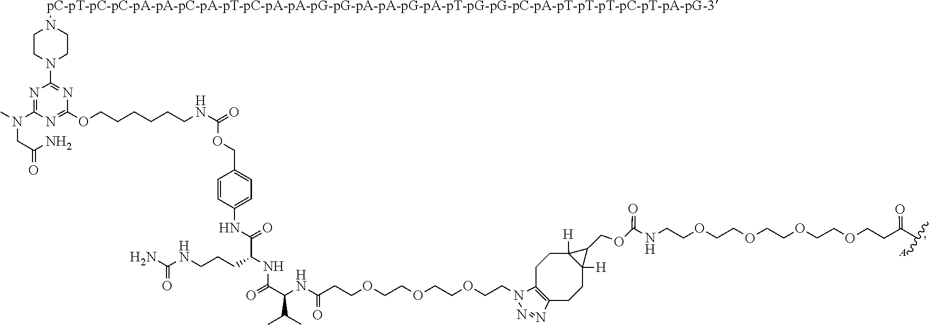

in which -pN indicates a base position of a phosphorodiamidate morpholino oligomer (PMO), R¹ is covalently linked to R² at attachment point A; wherein -p reflects a phosphorodiamidate linkage, wherein N corresponds to a nucleobase of adenine (A), cytosine (C), guanine (G), or thymine (T), such that the PMO has a nucleobase sequence of

(SEQ ID NO: 21)
CTCCAACATCAAGGAAGATGGCATTTCTAG.

18. The formulation of any one of embodiments 2-16, wherein each R¹ comprises formula:

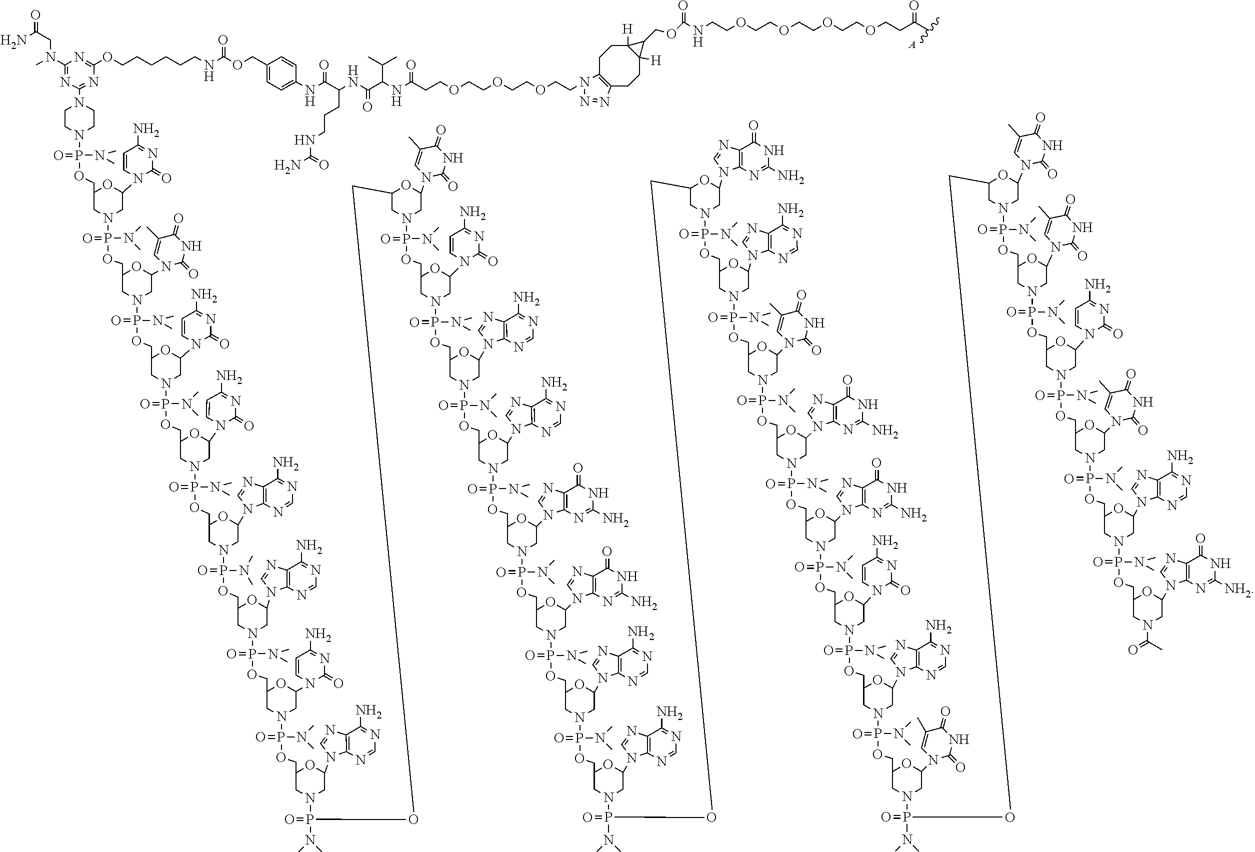

19. The formulation of any one of embodiments 1-18, wherein the complexes are present in the formulation at a concentration in the range of 10 mg/mL to 50 mg/mL.
20. A method of promoting expression or activity of a dystrophin protein in a subject, the method comprising administering to the subject the formulation of any one of embodiments 1-19.
21. The method of embodiment 20, wherein the dystrophin protein is a truncated dystrophin protein.
22. A method of treating a subject having a mutated DMD allele associated with Duchenne Muscular Dystrophy, the method comprising administering to the subject the formulation of any one of embodiments 1-19.
23. The method of embodiment 22, wherein the mutated DMD allele comprises a mutation amenable to exon 51 skipping.
24. The method of embodiment 22 or embodiment 23, wherein the mutated DMD allele comprises a frameshift mutation in exon 51.
25. A complex comprising a structure of the formula [R¹]ₙ₁—R², wherein
each R¹ comprises a group of the formula:

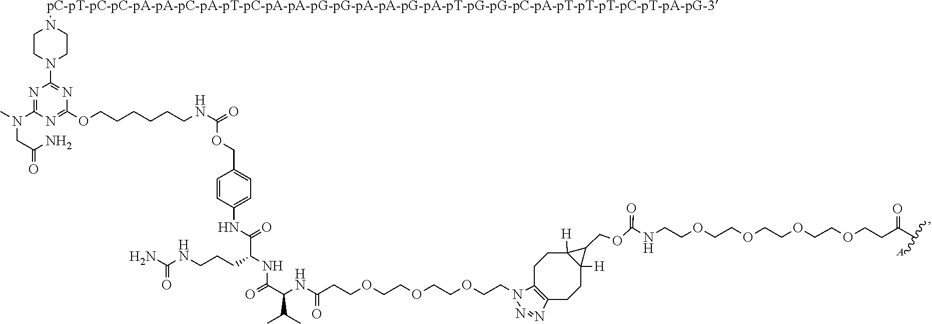

herein R² comprises a Fab, and wherein the Fab comprises a CDR-H1, a CDR-H2, a CDR-H3, a CDR-L1, a CDR-L2, and a CDR-L3 selected from Table 2, optionally wherein the Fab comprises a VH comprising the amino acid sequence of SEQ ID NO: 17 and a VL comprising the amino acid sequence of SEQ ID NO: 18, further optionally wherein the Fab comprises a heavy chain comprising the amino acid sequence of SEQ ID NO: 19 and a light chain comprising the amino acid sequence of SEQ ID NO: 20, wherein -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the oligonucleotide PMO has a nucleobase sequence of CTCCAACATCAAGGAAGATGGCATTTCTAG (SEQ ID NO: 21); wherein R¹ is covalently linked to R² at attachment point A; and wherein n1 is an integer of one or greater representing the number of instances of R¹, wherein each instance of R¹ is covalently linked to a different amino acid residue of the Fab, optionally wherein each different amino acid residue is a lysine.
26. A complex comprising a structure of the formula [R¹]ₙ₁—R², wherein
R¹ comprises a group of the formula:

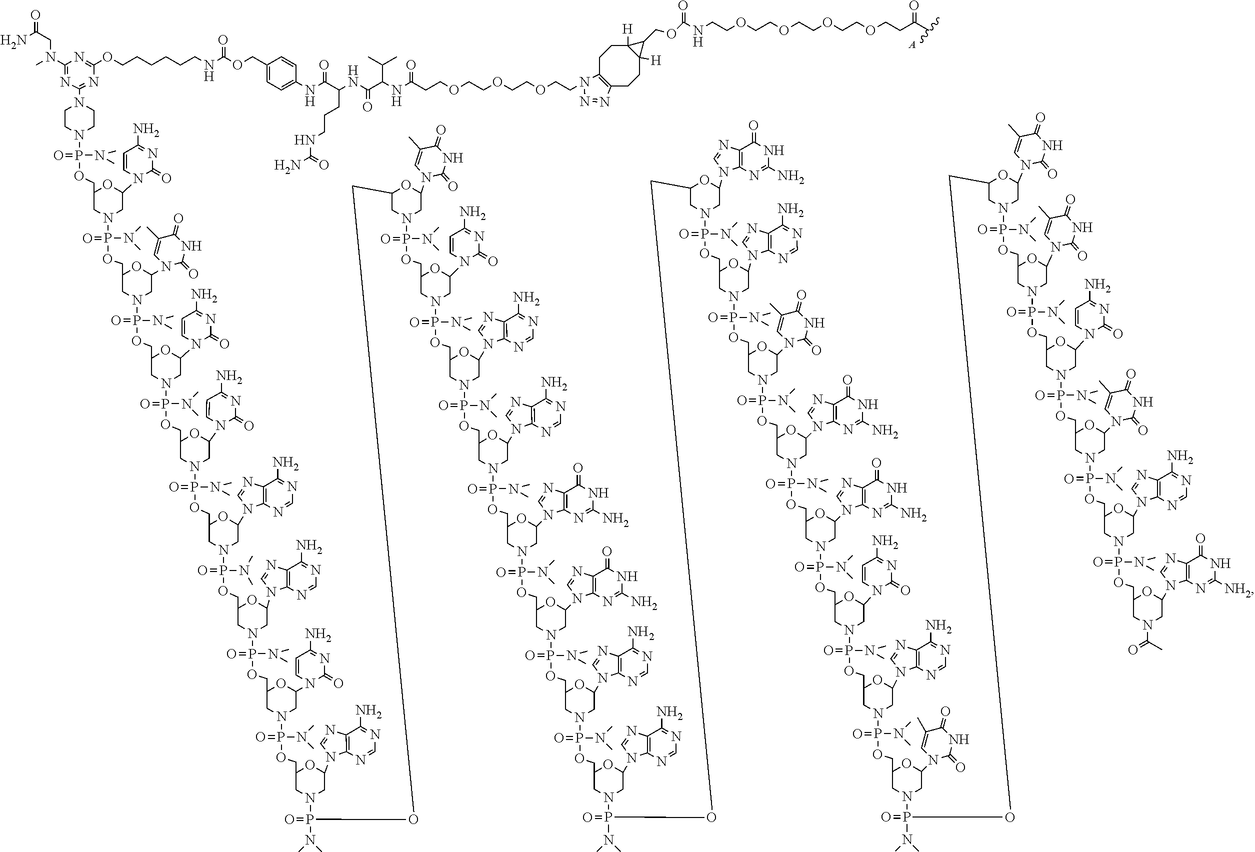

wherein R² comprises a Fab comprising a CDR-H1, a CDR-H2, a CDR-H3, a CDR-L1, a CDR-L2, and a CDR-L3 selected from Table 2, optionally wherein R² comprises a Fab comprising a VH comprising the amino acid sequence of SEQ ID NO: 17 and a VL comprising the amino acid sequence of SEQ ID NO: 18, further optionally wherein R² comprises a Fab comprising a heavy chain comprising the amino acid sequence of SEQ ID NO: 19 and a light chain comprising the amino acid sequence of SEQ ID NO: 20; wherein R¹ is covalently linked to R² at attachment point A; wherein n1 is an integer of one or greater representing the number of instances of R¹, wherein each instance of R¹ is covalently linked to a different amino acid residue of the Fab, optionally wherein each different amino acid residue is a lysine.
27. A formulation comprising a plurality of complexes of embodiment 25 or embodiment 26, and histidine at a concentration of 25 mM, and sucrose at a concentration of 10 w/v %, wherein the formulation is an aqueous solution and is at a pH of 6.0.
28. A lyophilized form of the formulation of embodiment 27.
29. A product produced by a process comprising lyophilizing the formulation of embodiment 27.
30. A frozen form of the formulation of embodiment 27.
31. A product produced by a process comprising freezing the formulation of embodiment 27.

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or (e.g., and) one or more modified nucleotides and/or (e.g., and) one or more modified internucleotide linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

1. A formulation comprising complexes that comprise a phosphorodiamidate morpholino oligomer (PMO) covalently linked to an anti-transferrin receptor 1 (TfR1) antibody, wherein the complexes are formulated with histidine and sucrose.

2. A formulation comprising complexes comprising a structure of formula: [R¹]ₙ₁—R², wherein
each R¹ independently comprises a group of the formula (Ia): 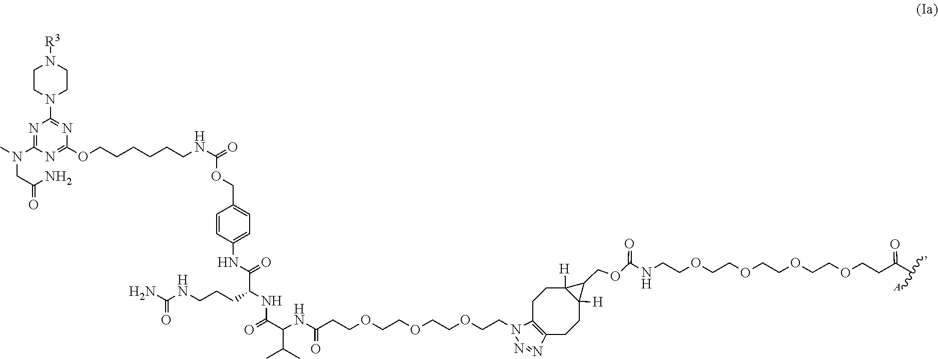
wherein:
R² comprises an antibody, and
R³ comprises a phosphorodiamidate morpholino oligomer (PMO);
wherein R¹ is covalently linked to R² at attachment point A; and
wherein n1 is an integer of one or greater representing the number of instances of R¹, wherein each instance of R¹ is covalently linked to a different amino acid residue of the antibody;
wherein the complexes are formulated with histidine and sucrose.

3. (canceled)

4. The formulation of any claim 2, wherein the formulation is in a lyophilized form, an aqueous solution, or a frozen solid form.

5. The formulation of claim 4, wherein the formulation is in an aqueous solution and wherein the histidine is present in the aqueous solution at a concentration in the range of 10 mM to 50 mM.

6. The formulation of claim 4, wherein the formulation is in an aqueous solution and wherein the sucrose is present in the aqueous solution at a concentration in the range of 5% to 15% weight per volume (w/v %).

7. The formulation of claim 4, wherein the formulation is in an aqueous solution and wherein the aqueous solution has a pH in the range of 5.0 to 7.0.

8. The formulation of claim 4, wherein the formulation is in an aqueous solution and wherein the histidine is present in the aqueous solution at a concentration of 25 mM and/or the sucrose is present in the aqueous solution at a concentration of 10 w/v % and/or the aqueous solution is at a pH of 6.0.

9. The formulation of claim 2, wherein the antibody is a Fab fragment, a full-length IgG, a Fab′ fragment, a F(ab′)₂ fragment, an scFv, or an Fv.

10-12. (canceled)

13. The formulation of claim 2, wherein the PMO comprises a nucleobase sequence that is 15-35 nucleotides in length.

14-17. (canceled)

18. The formulation of claim 2, wherein the complexes are present in the formulation at a concentration in the range of 10 mg/mL to 50 mg/mL.

19. A method of promoting expression or activity of a dystrophin protein in a subject, the method comprising administering to the subject the formulation of claim 2.

20. The method of claim 19, wherein the dystrophin protein is a truncated dystrophin protein.

21. A method of treating a subject having a mutated DMD allele associated with Duchenne Muscular Dystrophy, the method comprising administering to the subject the formulation of claim 2.

22. The method of claim 21, wherein the mutated DMD allele comprises a mutation amenable to exon 51 skipping.

23. The method of claim 21, wherein the mutated DMD allele comprises a frameshift mutation in exon 51.

24. A complex comprising a structure of formula (I): [R¹]ₙ₁—R², wherein
each R¹ comprises a group of the formula (Ia): 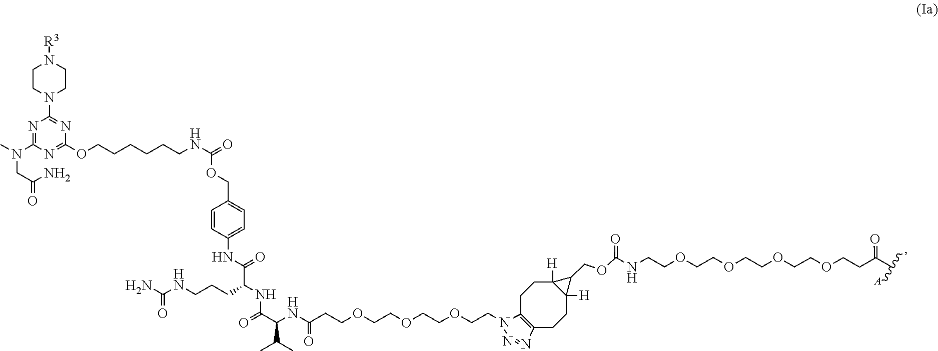
wherein R³ comprises a phosphorodiamidate morpholino oligomer (PMO);
wherein R² comprises a Fab,
wherein R¹ is covalently linked to R² at attachment point A; and wherein n1 is independently an integer of one or greater representing the number of instances of R¹ in each complex, wherein each instance of R¹ is covalently linked to a different amino acid residue of the Fab.

25-27. (canceled)


SEQUENCE LISTING (36 sequences)

SEQ ID NO: 1 - protein, 9 aa, synthetic construct
  REGION 1..9: note = Synthetic
GYSITSGYY

SEQ ID NO: 2 - protein, 7 aa, synthetic construct
  REGION 1..7: note = Synthetic
ITFDGAN

SEQ ID NO: 3 - protein, 12 aa, synthetic construct
  REGION 1..12: note = Synthetic
TRSSYDYDVLDY

SEQ ID NO: 4 - protein, 6 aa, synthetic construct
  REGION 1..6: note = Synthetic
QDISNF

SEQ ID NO: 5 - skipped

SEQ ID NO: 6 - protein, 9 aa, synthetic construct
  REGION 1..9: note = Synthetic
QQGHTLPYT

SEQ ID NO: 7 - protein, 6 aa, synthetic construct
  REGION 1..6: note = Synthetic
SGYYWN

SEQ ID NO: 8 - protein, 16 aa, synthetic construct
  REGION 1..16: note = Synthetic
YITFDGANNYNPSLKN

SEQ ID NO: 9 - protein, 10 aa, synthetic construct
  REGION 1..10: note = Synthetic
SSYDYDVLDY

SEQ ID NO: 10 - protein, 11 aa, synthetic construct
  REGION 1..11: note = Synthetic
RASQDISNFLN

SEQ ID NO: 11 - protein, 7 aa, synthetic construct
  REGION 1..7: note = Synthetic
YTSRLHS

SEQ ID NO: 12 - protein, 8 aa, synthetic construct
  REGION 1..8: note = Synthetic
GYSITSGY

SEQ ID NO: 13 - skipped

SEQ ID NO: 14 - protein, 8 aa, synthetic construct
  REGION 1..8: note = Synthetic
SYDYDVLD

SEQ ID NO: 15 - protein, 7 aa, synthetic construct
  REGION 1..7: note = Synthetic
SQDISNF

SEQ ID NO: 16 - protein, 6 aa, synthetic construct
  REGION 1..6: note = Synthetic
GHTLPY

SEQ ID NO: 17 - protein, 119 aa, synthetic construct
  REGION 1..119: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSS

SEQ ID NO: 18 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIK

SEQ ID NO: 19 - protein, 227 aa, synthetic construct
  REGION 1..227: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 20 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 30 bp, synthetic construct
  misc_feature 1..30: note = Synthetic
  source 1..30: mol_type = other DNA
ctccaacatcaaggaagatggcatttctag

SEQ ID NO: 22 - DNA, 30 bp, synthetic construct
  misc_feature 1..30: note = Synthetic
  source 1..30: mol_type = other DNA
ctagaaatgccatcttccttgatgttggag

SEQ ID NO: 23 - DNA, 13993 bp, Homo sapiens
  source 1..13993: mol_type = genomic DNA
tcctggcatcagttactgtgttgactcactcagtgttgggatcactcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcaggtcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctagaatggatttttcccggagccggaagccaggaggaaactacaccacactaaaacattgtctacagctccagatgtttctcattttaaacaactttccactgacaacgaaagtaaagtaaagtattggatttttttaaagggaacatgtgaatgaatacacaggacttattatatcagagtgagtaatcggttggttggttgattgattgattgattgatacattcagcttcctgctgctagcaatgccacgatttagatttaatgatgcttcagtggaaatcaatcagaaggtattctgaccttgtgaacatcagaaggtattttttaactcccaagcagtagcaggacgatgatagggctggagggctatggattcccagcccatccctgtgaaggagtaggccactctttaagtgaaggattggatgattgttcataatacataaagttctctgtaattacaactaaattattatgccctcttctcacagtcaaaaggaactgggtggtttggtttttgttgcttttttagatttattgtcccatgtgggatgagtttttaaatgccacaagacataatttaaaataaataaactttgggaaaaggtgtaaaacagtagccccatcacatttgtgatactgacaggtatcaacccagaagcccatgaactgtgtttccatcctttgcatttctctgcgagtagttccacacaggtttgtaagtaagtaagaaagaaggcaaattgattcaaatgttacaaaaaaacccttcttggtggattagacaggttaaatatataaacaaacaaacaaaaattgctcaaaaaagaggagaaaagctcaagaggaaaagctaaggactggtaggaaaaagctttactctttcatgccattttatttctttttgatttttaaatcattcattcaatagataccaccgtgtgacctataattttgcaaatctgttacctctgacatcaagtgtaattagcttttggagagtgggctgacatcaagtgtaattagcttttggagagtgggttttgtccattattaataattaattaattaacatcaaacacggcttctcatgctatttctacctcactttggttttggggtgttcctgataattgtgcacacctgagttcacagcttcaccacttgtccattgcgttattttctttttcctttataattctttctttttccttcataattttcaaaagaaaacccaaagctctaaggtaacaaattaccaaattacatgaagatttggtttttgtcttgcatttttttcctttatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccaagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaaatatatgccttactattgtattatagtactgctttactgtgtatctcaataaagcacgcagttatgttac

SEQ ID NO: 24 - DNA, 233 bp, Homo sapiens
  source 1..233: mol_type = genomic DNA
ctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaag

SEQ ID NO: 25 - DNA, 182 bp, Homo sapiens
  source 1..182: mol_type = genomic DNA
atgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacag

SEQ ID NO: 26 - DNA, 213 bp, Homo sapiens
  source 1..213: mol_type = genomic DNA
gctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcag

SEQ ID NO: 27 - DNA, 173 bp, Homo sapiens
  source 1..173: mol_type = genomic DNA
aatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagg

SEQ ID NO: 28 - DNA, 148 bp, Homo sapiens
  source 1..148: mol_type = genomic DNA
gcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaag

SEQ ID NO: 29 - DNA, 148 bp, Homo sapiens
  source 1..148: mol_type = genomic DNA
gctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaag

SEQ ID NO: 30 - DNA, 109 bp, Homo sapiens
  source 1..109: mol_type = genomic DNA
aggaagttagaagatctgagctctgagtggaaggcggtaaaccgtttacttcaagagctgagggcaaagcagcctgacctagctcctggactgaccactattggagcct

SEQ ID NO: 31 - DNA, 176 bp, Homo sapiens
  source 1..176: mol_type = genomic DNA
gaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagag

SEQ ID NO: 32 - DNA, 118 bp, Homo sapiens
  source 1..118: mol_type = genomic DNA
gcaacaatgcaggatttggaacagaggcgtccccagttggaagaactcattaccgctgcccaaaatttgaaaaacaagaccagcaatcaagaggctagaacaatcattacggatcgaa

SEQ ID NO: 33 - DNA, 212 bp, Homo sapiens
  source 1..212: mol_type = genomic DNA
ttgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaag

SEQ ID NO: 34 - DNA, 190 bp, Homo sapiens
  source 1..190: mol_type = genomic DNA
ggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaa

SEQ ID NO: 35 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 36 - DNA, 176 bp, Homo sapiens; identical to SEQ ID NO: 31
  source 1..176: mol_type = genomic DNA